(19) United States
(12) Patent Application Publication
(10) Pub. No.: US 2007/0087401 A1
(43) Pub. Date: Apr. 19, 2007

(54) Analysis of metabolic activity in cells using extracellular flux rate measurements

(76) Inventors: Andy Neilson, Groton, MA (US); Jay Teich, Berlin, MA (US); Min Wu, Carlisle, MA (US); David Ferrick, Carlisle, MA (US)

Correspondence Address: GOODWIN PROCTER LLP;PATENT ADMINISTRATOR, EXCHANGE PLACE, BOSTON, MA, 02109-2881, US

(21) Appl. No.: 11/545,714

(22) Filed: Oct. 10, 2006

Related U.S. Application Data
(63) Continuation-in-part of application No. 10/688,791, filed on Oct. 17, 2003.
(63) Continuation-in-part of application No. 11/486,440, filed on Jul. 13, 2006.
(60) Provisional application No. 60/724,669, filed on Oct. 7, 2005.

(51) Int. Cl. C12Q 1/02

(52) U.S. Cl. 435/29

(57) ABSTRACT

Disclosed are methods for non-destructively measuring in vitro the effect on cellular metabolism of the addition to animal cells in culture of a soluble molecule potentially capable of perturbing the biological state of the cells, such as a drug or drug candidate, a toxin, a ligand known or suspected to bind to a cell surface receptor, a nutrient, a cytokine, a growth factor, a chemokine, a metabolism inhibitor or stimulator. Also disclosed are methods for measuring cell viability, vitality, or quality, e.g., in anticipation of the execution of an experiment on the cells. The measurements are done by observing alteration in the rates of consumption or production of extracellular solutes related to aerobic and anaerobic cellular metabolism, such as oxygen, protons, nutrients, carbon dioxide, lactate, or lactic acid. The methods are particularly useful in drug discovery efforts, such as cancer drug discovery and searches for modulators of cellular metabolism.

### CROSS-REFERENCE TO RELATED APPLICATIONS

This application claims the benefit of and priority to U.S. Provisional Patent Application Ser. No. 60/724,669, filed Oct. 7, 2005, and is a continuation-in-part application of copending U.S. patent application Ser. No. 10/688,791, filed Oct. 17, 2003, entitled “Method and device for measuring multiple physiological properties of cells,” and published as US/2005/0054028, on Mar. 10, 2005, and copending U.S. patent application Ser. No. 11/486,440, filed Jul. 13, 2006, and entitled “Cell analysis apparatus and method.” The entire disclosures of each of these applications are incorporated by reference herein for all purposes.

### FIELD OF THE INVENTION

This invention relates to cell analysis methods, and more particularly to methods for probing animal cells, tissues and cellular organelles, such as mammalian cells in culture by measuring alterations in their metabolism, e.g., upon exposure to an environmental stress or a chemical such as a toxin, drug, drug candidate, ligand which interacts with a cell surface receptor, nutrient, growth factors, or naturally occurring molecule such as a hormone. It also relates to methods for testing the viability and vitality of cells in a culture preparatory to conducting an experiment on the cells. In this aspect, more particularly, the invention provides methods for profiling metabolic capacity, and or preference, e.g., as an assessment of cell quality, i.e., a measure of the metabolic health and potential of cells in culture.

### BACKGROUND

Living mammalian and other animal cells, tissues and certain cellular organelles consume nutrients and oxygen from the surrounding medium, and return metabolic byproducts, including ions, carbon dioxide, lactate, and various proteins, to this extracellular environment. Indeed, in a living cell there are thousands of chemical reactions, each coupled and progressing via a complicated network of inter and extra cellular processes. Most of these reactions are energy dependent and are coupled to metabolic pathways that yield energy.

Mammalian cells are able to consume a variety of nutrients to produce ATP, and are able to do so using multiple metabolic processes. This versatile energy production machinery is highly responsive to the impact of environmental changes and is regulated by the both energetic and biosynthetic needs of the cell. For example, when oxygen is unavailable, most cells quickly resort to anaerobic metabolism of glucose to maintain adequate ADP to ATP conversion rates. In fact, the complicated aerobic process for metabolism of nutrients, involving multiple steps of substrate conversion, the TCA cycle, and electron transport chain, is probably one of the most fragile functions performed by a cell.

In the absence of such nutrients in its immediate environment, or when otherwise appropriately modulated, a cell can exploit catabolism to provide the chemical energy necessary for its maintenance and/or biosynthetic needs. Catabolic processes break down large molecules such a polysaccharides, fat in adipose tissue, or proteins in order to use simple sugars, fatty acids, or amino acids as substrates for glycolysis or gluconeogenesis. These catabolic biochemical processes consume and produce chemical by-products such as oxygen (O₂), carbon dioxide (CO₂), protons (H+), glucose (C₆H₁₂O₆), lactic acid (C₃H₅O₃), ammonia (NH₃) and heat (AH). The rate of cellular uptake and excretion of these analytes can provide valuable information regarding the metabolic processes underway inside the cells.

As another example, the amount of CO₂ produced by a cell is a key indicator of metabolic processes. The ratio of CO₂ production to O₂ utilization is termed the Respiratory Quotient (RQ) and is a critical indicator of substrate utilization (RQ=CPR/OCR). RQ values for common substrates are: glucose: 1.0; protein: 0.82; fat: 0.7; ethanol: 0.67

As still another example, the difference between total extracellular proton flux, as derived by measurement of the extra cellular acidification rate (ECAR) and proton flux as derived by CO₂ would be an indirect method for determining lactate production.

A third pathway is that of the pentose phosphate pathway (also called hexose monophosphate (HMP) shunt) which serves to generate NADPH and the synthesis of pentose (5-carbon sugars). There are two distinct phases in the pathway: the first is the oxidative phase, in which NADPH is generated; and the second is the non-oxidative synthesis of 5 carbon sugars. The pathway is one of the three main ways mammalian cells create reducing molecules to generate ATP while preventing oxidative stress, accounting for approximately 10% of NADPH production in humans. Glucose is a requirement for the production of CO₂ through this pathway, and therefore it should be possible to determine the relative amount of activity through this pathway by comparing the amount of CO₂ produced by cells that have access to glucose versus cells that have access to an alternate carbon source such as glutamine.

Knowledge of the metabolic pathways and rates of ATP turnover and uncoupled metabolism employed by cells can be useful in developing new therapies to treat cancer, metabolic disease, and other diseases, and also to screen for unexpected or adverse effects of new drug candidates. Metabolic rate and pathway information can also be useful for assessing the health or status of cells.

Anti-cancer drug discovery is an area of particular research interest that could benefit from better and more detailed metabolic information. Research has consistently shown a difference in the metabolic mechanisms of cancer cells relative to their untransformed counterparts. More than eighty years ago, Otto Warburg observed that many cancer cells uniquely rely on glycolysis in the presence of oxygen, a phenomenon known as aerobic glycolysis. Many current cancer drugs, including gefitnib, imatinib, topotecan, tamoxifen, and cisplatin, target the pathways that control glucose metabolism. A better understanding of the metabolic properties of cancer cells could lead to new therapies that target unique weaknesses, such as limited aerobic respiration capacity.

Unfortunately, few methods exist to measure the metabolic properties of mammalian cells. One method, using sampling of headspace gas in a closed vessel containing cells was described by Guppy (J Cell Phys 170:1-7 (1997)). Another method, using a flow channel measurement system equipped with a waste stream oxygen sensor, was described by Beeson (Anal Biochem 304, 139-146 (2002)). Neither system was able to produce data of sufficient quality to analyze drug-induced metabolic behavior changes within a typical effective dose range.

Copending U.S. application Ser. No. 10/688,791, filed Oct. 17, 2003, titled “Method and device for measuring multiple physiological properties of cells,” published as 20050054028, and copending U.S. application Ser. No. 11/486,440 filed Jul. 13, 2006, entitled “Cell analysis apparatus and method” (the disclosures of which are incorporated herein by reference) discloses novel apparatus and methods for detecting in real time, conveniently, and with significant precision extracellular constituents present in media surrounding cells in culture.

### SUMMARY OF THE INVENTION

The present invention provides an assay system of broad applicability based on the ability to measure both aerobic and anaerobic components of cellular metabolism. Cells respond to conditions in their environment and to internal growth/differentiation programs by (among many other ways) accelerating, slowing, or altering their metabolism or the degree of exploitation of one metabolic pathway over one or more others. It is now possible as disclosed herein to probe the metabolism of cells in culture, and to measure multiple extracellular concentration changes of components involved in metabolism, preferably simultaneously. Accordingly, this invention provides ways of assessing the viability, vitality, metabolic profile, and quality of cells in culture, and ways to measure a cell culture's response to various stimuli.

In one aspect, the invention provides a method for animal cell culture analysis comprising the steps of incubating the cells in a medium disposed in at least one of a plurality of wells in a multi-well plate; adding to the medium to bring into contact with the cells a substance potentially capable of altering cellular metabolism; and measuring in a cell medium in a well the rate of change in concentration of both an extracellular solute which is a component of cellular aerobic metabolism and an extracellular solute which is a component of cellular anaerobic metabolism. The cells under analysis may be, for example, primary animal cells, such as cells growing on a surface in a well, neoplastic cells, or cells disposed in suspension. Furthermore, cell organelles such as mitochondria may be examined, and tissues comprising multiple cells, optionally present together with extracellular matrix, may be examined. Use of the therm “cells” in the appended claims is intended to include sub-parts of cells and sampled tissue. The substance added to the medium may be a drug or drug candidate, a toxin, a ligand known or suspected to bind to a cell surface receptor, a nutrient, cytokine, chemokine, or antibody—essentially any soluble molecule potentially capable of perturbing the biological state of the cells. Preferably, measurements are conducted substantially simultaneously in a well.

The method may comprise the additional steps of measuring, in the cell medium prior to addition of the substance, or in the medium of a cell culture in a well separate from the cells under analysis, the rate of change in concentrations of the extracellular solutes which are components of cellular aerobic and anaerobic metabolism to establish control or baseline values. The method may also comprise incubating the cells in the presence of the substance for a predetermined time interval prior to measuring the rates of change, or using one of a variety of established methods to insert, delete, or modify one or more genes within said cells prior to analysis. The method may also comprise measuring, in the medium of a cell culture in a well separate from the cells under analysis and treated differently than the cells under analysis, either or both the rate of change in concentration of extracellular solutes which are respectively components of cellular aerobic and anaerobic metabolism, and then comparing the measurements of the rates of change in the separate cell cultures. In still another aspect, the method may feature the steps of adding to the medium in separate cultures of the same cells in different wells different concentrations of the substance potentially capable of altering cellular metabolism, and measuring the rates of change in the cell medium in the separate cultures. Alternatively, the same data may be obtained by making multiple serial additions of the substance to increase its concentration in the media in a single well serially, and making measurements after each addition. Also, the method may be practiced by measuring in the cell medium the rates of change in concentration of oxygen, carbon dioxide, protons, etc., at different times to obtain a temporal profile of the effect of the substance on said cells.

In a preferred embodiment, the method comprises adding a fatty acid to a well to assess a characteristic of fatty acid metabolic activity of the cell culture. In still another aspect, the method may feature the steps of incubating the cells in cell media containing a substance suspected to alter the rate of fatty acid metabolic activity of the cell culture prior to measurement. A further improvement to this method includes the additional step of adding a known inhibitor of fatty acid transport or oxidation in order to more specifically determine the effect of the substance.

The measured component of cellular aerobic metabolism is preferably extracellular oxygen, and the measurement is oxygen consumption rate (OCR). The measured component of cellular anaerobic metabolism is preferably extracellular proton concentration (extracellular acidification rate −ECAR), or carbon dioxide production rate (CPR). Lactic acid production rate, or lactate production rate can also be used. Other molecules absorbed or secreted by animal cells and related to metabolic activities also may be exploited. The method may comprise the steps of incubating in parallel plural cultures of animal cells in plural wells, adding to the media in different wells different substances or different concentrations of the same substance, and measuring the rate of change in plural wells.

Preferably, as disclosed herein and in greater detail in pending U.S. application Ser. No. 11/486,440 filed Jul. 13, 2006, entitled Cell analysis apparatus and method, and in published US application 20050054028, the step of measuring the rate of change in concentration in the cell media comprises the step of temporarily reducing the volume of medium in a well containing a cell culture to produce a temporary small volume of media about the culture and to increase the sensitivity of solute concentration changes, and preferably detecting the changes using a solute concentration sensitive fluorescent probe.

In yet another embodiment, the invention provides a method for analysis of cell culture quality comprising the steps of measuring in a cell medium the rate of change in concentration of both an extracellular solute which is a component of cellular aerobic metabolism and an extracellular solute which is a component of cellular anaerobic metabolism, and comparing the measured rates of change to a standard informative of known cell culture respiration rates, thereby to assess the respiratory capacity of the culture as a measure of cell vitality and cell quality. This cell quality measurement method may comprise comparing the measured rates of change to rates measured in a culture comprising a known number of healthy cells of the same cell type or of a cell type having comparable metabolic characteristics to the cells under quality assessment. Preferably, this method comprises seeding cells at a predetermined density in a test well prior to the measuring step thereby to enable direct comparison of the measured rates of change to a standard. This method does much more than take a measurement indicative of whether the cells in a culture are alive, as it can measure metabolic rate; measure relative contribution of aerobic (oxidative phosphorylation) versus anaerobic (glycolysis) processes for generation of ATP; measure adherent cells in a microplate; or measure suspended cells in a microplate. Furthermore, the quality assessment is non destructive, and therefore the planned experiment on the cells can be conducted after assessing cell vitality and quality.

In a related aspect, the invention permits the scientist to obtain data indicative of respiratory (or metabolic) capacity of a cell culture without cell counting. This is done by measuring a basal metabolic rate or rates (i.e., rates of change of OCR, ECAR etc.), before the addition of any metabolism altering substance, followed by adding to the culture a drug that increases metabolism, and then repeating the measurement. A class of substances suitable for this purpose are drugs known to uncouple the TCA cycle within a cell, thereby producing waste heat in lieu of providing energy via ADP to ATP conversion. The increased respiration rate is indicative directly of metabolic capacity, and the ratio can be used as such a measure independent of the actual amounts of cells in the test well in which the measurements were made. This eliminates the need for cell count to normalize data, and can be particularly valuable when cell number is different in various wells or when cells proliferate during the experiment (particularly cancer cells).

In its various applications, the methods of the invention enable data collection based on experiments such as the following.

In certain embodiments, methods of the invention include profiling the metabolic function of living cells by comparing measurements of the extracellular flux rate of at least two analytes selected from gasses, ions, nutrients and byproducts of or component necessary for metabolism. The method may further include the use of the measured flux rates with a model of cellular metabolic processes to infer information regarding the total metabolic rate, ATP turnover and uncoupled heat production, and specific metabolic pathways within the cell. These methods may be used to assess aerobic versus anaerobic metabolism, including situations: where at least one analyte is sensitive to aerobic metabolism and at least one second analyte is sensitive to anaerobic metabolism; where one analyte is sensitive to either aerobic or anaerobic metabolism alone and a second analyte is insensitive to both; where an analyte sensitive to anaerobic metabolism is O₂, CO₂, and the like; where an analyte sensitive to anaerobic metabolism is proton flux (pH change), lactate, and the like; or where two analytes, one sensitive to aerobic and another sensitive to anaerobic metabolism can be used to calculate the amount of ATP generated per unit of time and the percent uncoupled metabolism per unit time.

The methods may be used for the purpose of understanding the relative contribution in a cell culture under a given set of conditions of glycolysis versus oxidative phosphorylation, or understanding total metabolic rate such as, but not limited, to ATP turnover and uncoupled heat production.

The methods may be used to measure a change in cellular metabolic function induced by exposure to a toxin such as one that induces necrosis or apoptosis; or a toxin that arrests proliferation or impairs nutrient transport, conversion, or mitochondrial function. Alternatively, or in addition, the methods may be used to measure a change in cellular metabolic function induced by exposure to a drug or drug candidate, genetic modification that induces necrosis, apoptosis, or metabolic impairment; or environmental stress that induces necrosis, apoptosis, or metabolic impairment.

Other aspects of the invention include a method for determining the effect of candidate drug compounds on anaerobic glycolysis in cancer cells by measuring the relative flux rates of analytes that are sensitive to glycolysis versus insensitive to glycolysis or sensitive to oxidative phosphorylation. Cells may be treated, measured, and compared to untreated cells. Treatment may include drug exposure, genetic modification (RNAi and the like), and environmental changes (pH, temperature, radiation and the like). The baseline may be measured, the cells treated, and the measurement repeated one or many times.

In another aspect, the invention includes a method for non-invasively assessing the magnitude of fatty acid oxidation (FAO) within living cells by measuring the relative flux rates of analytes that are sensitive to versus insensitive to FAO, or sensitive to FAO versus sensitive to metabolism of other nutrients including glucose and amino acids. The methods may be used with analytes sensitive to FAO, i.e., O₂. Analytes insensitive to FAO include protons and lactate.

In addition, mitochondrial DNA replication is less sophisticated than nuclear DNA, and is generally more susceptible to disruption. The relative rate of utilization of aerobic metabolism can be a sensitive indicator of mitochondrial dysfunction induced by environmental or genetic damage. The measurement of extracellular metabolic flux rates also comprises a novel and useful method for assessing mitochondrial function in cells.

### BRIEF DESCRIPTION OF THE DRAWINGS

The objects and features of the invention may be more fully understood by reference to the drawings described below in which:

FIG. 1 is a schematic illustration of a complete measurement system and apparatus in accordance with one embodiment of the invention;

FIGS. 2A and 2B are upright and inverted (respectively) exploded perspective views of a multiwell plate and a covered cartridge adapted to mate with the multiwell plate in accordance with one embodiment of the invention;

FIGS. 3, 4 and 5 together present a schematic, isolated, partial cross-sectional, exploded view of one region of an embodiment of the apparatus of the present invention, illustrating (FIG. 3) a portion of a sensor probe structure including an internal optical fiber bundle for light transmission to and from fluorescent sensor spots, the probe structures being inserted through a pneumatic multiplexer; the cartridge (FIG. 4—see FIGS. 2A and 2B) illustrating spots of fluorescent sensors disposed on an outside of a sleeve defining an aperture for receiving the portion of a sensor probe of FIG. 3, and two ports adapted to deliver a test fluid to a single well of the multiwell plate; and a single well of the multiwell plate (FIG. 5, see FIGS. 2A and 2B);

FIGS. 6a and 6b are schematic cross-sectional views of the probe structure, cartridge portion, and single well of FIGS. 3, 4, and 5 in a partially raised (mix or equilibrate) position and in a lowered (data gathering) position;

FIG. 7 is a schematic top view of four layers of a microfabricated pneumatic multiplexer, a portion of which is shown in FIG. 3;

FIG. 8 is a schematic cross-sectional view of three regions each comprising a probe, a portion of a cartridge, and a well in combination with the pneumatic multiplexer of FIG. 7;

FIG. 9 is a schematic view of a probe submerged in the liquid media contained within a microplate well and showing the use of gas pressure to deliver a drug to the media;

FIG. 10 is a block diagram of an embodiment of a system according to the invention;

FIG. 11 is a graph of OCR and ECAR versus time illustrating the effect on metabolism of the addition of various metabolism affecting drugs, and is discussed in Example 1;

FIG. 12 is a bar graph showing that an independent measure of ATP production was confirmatory of the data shown in FIG. 11, and that the cells remained viable;

FIG. 13 is a bar graph showing a comparison of OCR under different parallel conditions of two cancer cell types. It is discussed in Example 2;

FIG. 14 is a graph illustrating OCR response to increasing doses of doxorubicin on H460 cells. It is discussed in Example 3;

FIG. 15 is a graph of percent and number of viable cells as determined by trypan blue counting. It is discussed in Example 4;

FIG. 16 is a graph of OCR and ECAR versus imatinib concentration. It is discussed in Example 4;

FIG. 17A, FIG. 17B, and FIG. 17C are graphs of percent of basal metabolic rate for cells pretreated with imatinib versus concentration of three metabolism modifiers. They are discussed in Example 5;

FIG. 18 is an illustration of specific steps of a metabolic pathway;

FIGS. 19A-E are graphs of percent change in OCR and ECAR relative to baseline in response to five different compound treatments. They are discussed in Example 6;

FIG. 20 is a graph of OCR and ECAR versus DMOG concentration. It is discussed in Example 7;

FIG. 21 is a data comparison table discussed in Example 7;

FIG. 22 is a graph of OCR and ECAR versus topotecan concentration based on data gathered from an experiment on C₄₋₂ cells. It is discussed in Example 7;

FIG. 23 is a graph of percent of basal metabolic rate versus compound concentration discussed in Example 8;

FIG. 24 is a graph of percent of basal metabolic rate versus compound concentration discussed in Example 8;

FIG. 25 are graphs of percent of basal metabolic rate versus oxamate concentration and versus GLUT-1 concentration discussed in Example 9;

FIG. 26 depicts four graphs of percent of basal metabolic rate versus concentration of four metabolism influencing substances for three different cell types discussed in Example 11;

FIG. 27 is a bar graph showing percent response from basal metabolic rate in ECAR, OCR and the ratio in an experiment testing fatty acid metabolism as reported in Example 12;

FIG. 28 is a bar graph showing percent response from basal metabolic rate in ECAR, OCR and their ratio in an experiment testing fatty acid metabolism as reported in Example 13;

FIG. 29 is a bar graph showing percent response from basal metabolic rate in ECAR, OCR and their ratio in an experiment testing fatty acid metabolism as reported in Example 14;

FIG. 30 depicts bar graphs of OCR and ECAR illustrative of the fidelity of measurements of cell quality. It is discussed in Example 15;

FIG. 31 depicts bar graphs of percent response from basal metabolic rate and Trypan blue-determined cell viability in an experiment described in Example 17;

FIG. 32 is a bar graph of respiration rate for two cancer cell lines in the presence of rotenone, discussed in Example 18;

FIGS. 33A-33D are graphs depicting attenuated OXPHOS capacity of H460 and A469 cells, in the presence and absence of glucose;

FIGS. 34, 35, and 36 are graphs of OCR, ECAR and CPR versus time discussed in Example 19.

### DETAILED DESCRIPTION

The description is organized by first explaining the nature, structure, and various modes of operation of the currently preferred apparatus for gathering the data upon which the methods of the invention are based. Next the protocols of the invention and certain rationale's for them are described generally, as well as the fundamental biology and biochemistry on which the methods of the invention are based. Then, a number of specific analysis protocols which variously embody the claimed methods are disclosed by way of non-limiting examples.

A. Apparatus and Techniques for Measuring Changes in Concentration of Multiple Extracellular Solutes in a of a Microplate Containing a Cell Culture in a Medium

The practice of the invention requires the measurement of plural extracellular solutes adjacent living cells disposed, for example, in a well of a multiwell microplate. The currently preferred methods and means for implementing this function are described in detail below and in the aforementioned copending applications. Many other methods may be used to gather such data exploiting known techniques, but these necessarily will be tedious and error prone as compared with the preferred apparatus, now available commercially from Seahorse BioScience, Inc., of Billerica, Mass., under the trademark CellDoctor™.

The apparatus provides a low cost per test, high throughput cellular assay system ideal for gathering data in the practice of the invention disclosed herein. It includes an array of submersible sensors that enables fast sensor stabilization within multiple cell-containing wells simultaneously, thereby increasing measurement throughput. It also includes compound storage and delivery apparatus, a pneumatic multiplexer, structure for adding fluids to subsets or all of multiple wells simultaneously, and sensor structure permitting non destructive measurement of the effect of addition of exogenous fluid to respective wells, in combination with the ability to make and repeat measurements rapidly. Furthermore, the apparatus is designed to exploit a cartridge structure which permits repeated use of the apparatus for disparate cellular assays without requiring intermediate cleaning, and while eliminating the possibility of cross contamination between tests. Still further, the apparatus provides software for designing and implementing multi-well cellular assays run in parallel, and for receiving and analyzing the generated data that is intuitive and easy to use, permits multiple scientists to design and execute multiwell parallel assays during the same time period, and preferably is based on a spreadsheet program of the type well understood by scientists and easily integrated with sophisticated LIMS systems.

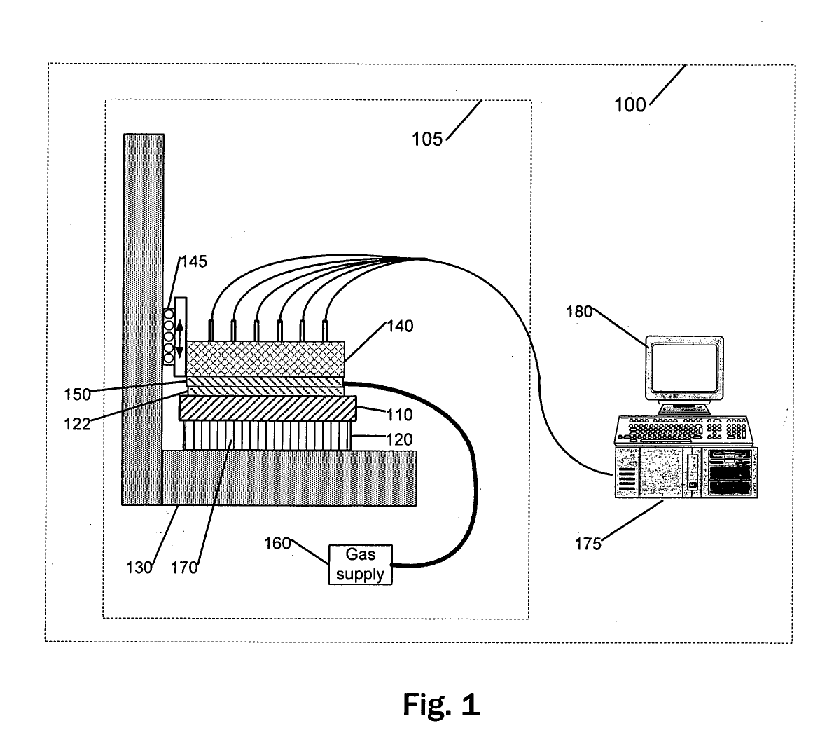

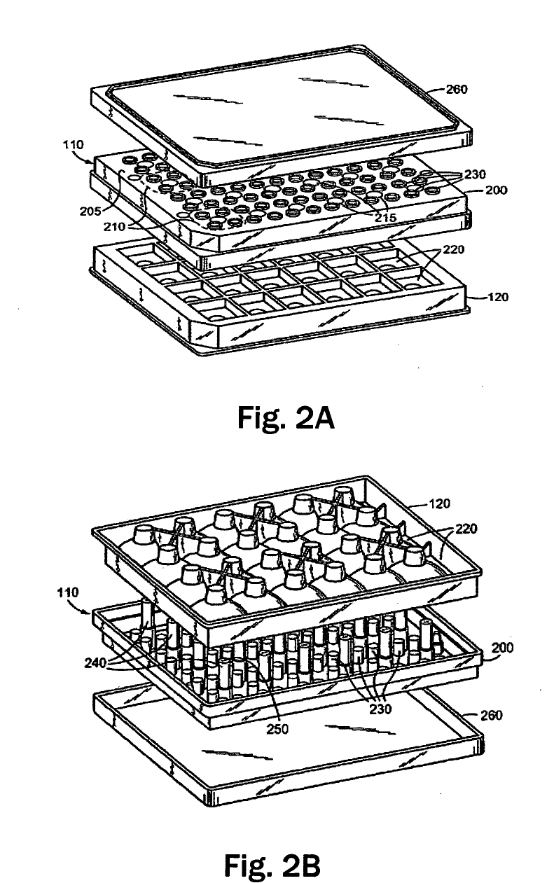

Referring to FIG. 1, the apparatus 100 is illustrated schematically. It comprises a compound storage and delivery apparatus in a housing 105 (shown in dashed lines) that includes a cartridge 110 defining a plurality of apertures for receiving sensor structures and a plurality of fluid ports (shown in detail in FIGS. 2A and 2B) compliantly mounted, and a stage or base 130 adapted to receive a multiwell plate 120, e.g., a cell culture plate. The cartridge 110 is disposed above, and adapted to mate with, the multiwell plate 120. The cartridge 110 optionally is held by a cartridge holder 122 adapted to receive the cartridge 110. The compound storage and delivery apparatus 105 also includes a mounting block 140, which can reciprocate as shown by the double headed arrow, preferably powered by a motor (not shown), including an elevator mechanism 145. The elevator mechanism 145 is adapted to move the cartridge 110 relative to the stage 130, or well plate 120. The mounting block includes a gas multiplexer 150 attached to a pressure source, e.g., gas supply or gas reservoir 160. The gas supply 160 is in fluid communication with the cartridge, and is used to impel the delivery of test fluid from a port in the cartridge to a well in the multiwell plate 120 as disclosed below. A plurality of sensor probes 170 are adapted for insertion into the plurality of apertures in the cartridge 110, and may be used to gather data indicative of the state of cells disposed in wells in the multiwell plate 120.

The compound storage and delivery apparatus 105 is controlled by a controller 175, that may be integrated with a computer 180, that may control the elevator mechanism, the multiplexer, and the pressure source. The controller 175 may, thereby, permit delivery of a test fluid from a port to a corresponding well when an associated sensor is disposed in the well.

FIGS. 2A and 2B illustrate the currently preferred form of the cartridge 110 and microplate 120, and how they relate to each other The cartridge is a generally planar element comprising a frame 200 made, e.g., from molded plastics. Planar surface 205 defines a plurality of regions 210 that correspond to, i.e., register with, a number of the respective openings of a plurality of wells 220 defined in the multiwell plate 120. Within each of these regions 210, in the depicted embodiment, the planar element defines first, second, third, and fourth ports 230, which serve as test compound reservoirs, and a central aperture 215 to a sleeve 240. Each of the ports is adapted to hold and to release on demand a test fluid to the respective well 220 beneath it. The ports 230 are sized and positioned so that groups of four ports may be positioned over the wells 220, and test fluid from any one of the four ports may be delivered to a respective well 220. In other embodiments the number of ports in each region may be less than four or greater than four. The ports 230 and sleeves 240 may be compliantly mounted relative to the microplate 120 so as to permit it to nest within the microplate by accommodating lateral movement. The construction of the microplate to include compliant regions permits its manufacture to looser tolerances, and permits the cartridge to be used with slightly differently dimensioned microplates. Compliance can be achieved, for example, by using an elastomeric polymer to form planar element 205, so as to permit relative movement between frame 200 and the sleeves and ports in each region.

Each of the ports 230 may have a cylindrical, conic or cubic shape, open through planar element 200 at the top, and closed at the bottom except for a small hole, i.e., a capillary aperture, typically centered within the bottom surface. The capillary aperture is adapted to retain test fluid in the port, e.g., by surface tension, absent an external force, such as a positive pressure differential force, a negative pressure differential force, or possibly a centrifugal force. Each port may be fabricated from a polymer material that is impervious to test compounds, or from any other solid material. When configured for use with a multiwell microplate 120, the liquid volume contained by each port may range from 500 μl to as little as 2 μl, although volumes outside this range are contemplated.

In the depicted embodiment, multiwell plate 120 has 24 wells. The number of wells 220 in a plate may vary from 1 to several thousand. In other embodiments, a single well of nearly any size may be fabricated, or multiple wells may be fabricated, or multiple wells may be fabricated in a one- or two-dimensional arrangement. In one embodiment, a two-dimensional pattern of wells corresponding to the pattern and dimensions of a microplate, as described by the Society for Biomolecular Screening standards for microplates (“SBS-1 Footprints” and “SBS-4 Well Positions,” both full proposed standards updated May 20, 2003), and containing a total of 12, 24, 96, 384, 1536, or any other number of individual wells may be fabricated.

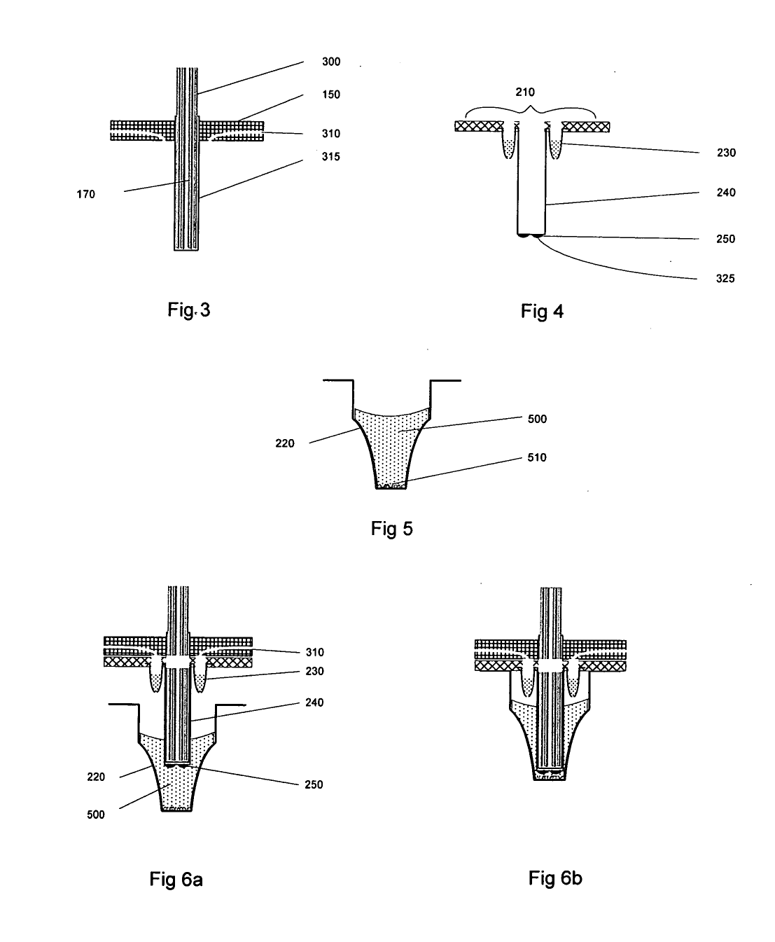

Referring to FIG. 2b, in each region of the cartridge 110, disposed between and associated with one or more ports 230, is a submersible sensor sleeve or barrier 240, adapted to be disposed in the corresponding well 220. Sensor sleeve 240 may have plural sensors 250 disposed on a lower surface 255 thereof for insertion into media in a well 220. One preferred example of a sensor for this purpose is a fluorescent indicator, such as an oxygen-quenched fluorophore, embedded in an oxygen permeable substance, such as silicone rubber. The fluorophore has fluorescent properties dependant on the presence and/or concentration of a constituent in the well 220 (in this example oxygen concentration). Other types of known sensors may be used, such as electrochemical sensors, Clark electrodes, etc. Sensor sleeve 240 may define an aperture and an internal volume adapted to receive a sensor. Examples of the types of sensors that may be used are described below with reference to FIG. 3.

The cartridge 110 may be attached to the sensor sleeve, or may be located proximal to the sleeve without attachment, to allow independent movement. The cartridge 110 may include an array of compound storage and delivery ports assembled into a single unit and associated with a similar array of sensor sleeves.

The apparatus may also feature a removable cover 260 for the cartridge 110 or for multiwell plate 120. The configuration of cartridge 110 as a cover for multiwell plate 120 may help prevent evaporation or contamination of a sample or media disposed in wells 220. The cover 260 may also be configured to fit over the cartridge 110 thereby to reduce possible contamination or evaporation of fluids disposed in the ports 230 of the cartridge 110.

Referring also to FIG. 3 through 6B, details of preferred relationship of parts is illustrated. FIG. 3 shows a fixed (preferably not part of the cartridge and reusable) sensor probe structure 170 configured to fit within the sensor sleeve 240. The sensor probe structure 170 includes a rigid outer tube 315 made from, e.g., stainless steel. Optical fibers 300 are disposed within the tube 315, and are configured to stimulate one or more fluorophores 250 disposed on a light transmissive outside lower wall portion 325 of sensor sleeve 240 and to receive fluorescent emissions from the fluorophore through the wall portion. When the probe is in its down position, it forms a reduced media test volume in each well, as shown, for example, in FIG. 6B. As an alternative (not shown) probe sleeve 240 may comprise an annular wall portion extending below portion 325 which defines the reduced test volume. The sensor probe structure and fluorophore may be configured to read optical density, luminescence, phosphorescence, or, preferably, fluorescence. In an alternative embodiment (not shown) the sensor probe structure 170 may be a self contained sensor which gathers data from a well through a signal transmissive bottom wall of the sleeve 240, or directly through an open bottom on the sleeve, preferably sealed to the probe.

Various types of sensors can be utilized depending on the analysis to be performed and its selected configuration, including oxygen sensors, such as oxygen-quenched fluorescent sensors, pH sensors, including fluorescent sensors, ISFET and impedance sensors using electrodes coupled through bottom wall 325 of sleeve 240, CO₂ sensors, including bicarbonate buffer coupled and ammonium dye coupled fluorescent sensors as well as other CO₂ sensors; various ion and small molecule sensors; large molecule sensors including surface plasmon resonance sensors and sensors exploiting the principle of Wood's anomaly; acoustic sensors; and microwave sensors. In certain embodiments, a conventional plate reader may be used.

Preferred sensors are fluorophores. Many fluorescent sensing compounds and preparations are described in the art and many are available commercially from, for example, Molecular Probes Inc and Frontier Scientific, Inc. The currently preferred oxygen sensor is a fluorophore with the signal inversely proportional to oxygen concentration such as a porphyrin or rhodamine compound immobilized as a particle or homogenously distributed in an oxygen permeable polymer, e.g., silicone rubber. The currently preferred compound is porphyrin. The currently preferred pH sensor is a fluorescent indicator dye, fluorescein, whose signal decreases upon protonation of the dye, and which is either entrapped in a particle that is suspended in a carrier polymer, or covalently attached to a hydrophilic polymer. The currently preferred CO₂ sensor employs a pH sensitive transducer, with the fluorescence being indirectly modulated by the production of carbonic acid due to reaction of carbon dioxide with water. See, e.g. O. S. Wolfbeis, Anal. Chem. 2002, 74, 2663-2678. A fluorophore that detects glucose also can be used, such as one based on a non-enzymatic transduction using a boronic probe that complexes with glucose, resulting in a charge transfer that modulates the fluorescence of the probe, or an enzymatic glucose transducer that couples a glucose oxidase to a fluorescent oxygen sensor, with the binding and oxidation of glucose resulting in a quantitative modulation of the oxygen sensor. One can employ a fluorophore or other type of sensor sensitive to biological molecules such as, for example, lactate, ammonia, or urea. A lactate sensor can be based on an enzymatic sensor configuration, with lactate oxidase coupled to a fluorescent oxygen sensor, and with the binding and oxidation of lactate resulting in a quantitative modulation of the oxygen sensor. An ammonia or ammonium ion sensor can be configured with immobilization of a protonated pH indicator in a hydrophobic, gas permeable polymer, with the fluorescence output quantitatively modulated by reaction with transient ammonia. A urea sensor can be based on an enzymatic sensor configuration, with urease coupled to a fluorescent ammonia transducer, and with the binding and reduction of urea to ammonia, resulting in modulation of the ammonia sensor fluorescence.

In the illustrated embodiment, the fixed sensor probe 170 is attached to and extends orthogonally from the pneumatic multiplexer 150. Other sensor configurations will be apparent to those skilled in the art. For example, probes may be disposed on a wall within the well under examination, or on a bottom, translucent surface of a well.

Air channels 310 are defined within the pneumatic multiplexer 150 and are positioned to feed drug wells or ports 230 when the elongated neck of the fixed sensor probe 315 is fitted within with the sleeve 240. The pneumatic multiplexer 150 serves to deliver compressed gas to a plurality of ports (see FIG. 6a) from a single source that may be controlled by an electrical or mechanical gas regulator or valving. Other types of pneumatic, mechanical or hydraulic pressure actuators may be used. For example, the actuator may be a piston within a sleeve, as described in U.S. Pat. No. 4,498,510 to Minshew et al., or a controlled gas supply as described in U.S. Pat. No. 4,461,328 to Kenney, or any other suitable means for ejecting liquid test compound from the bottom of the port 230 using an extrinsic force.

The use of a pneumatic multiplexer may be preferable for the sake of simplification and reduction of the number of components that supply compressed gas to the apparatus. The currently preferred pneumatic multiplexer 150 is discussed in greater detail below.

Referring to FIG. 4, a region 210 of the cartridge 110 includes first and second ports 230. In use, a test compound such as a drug, drug candidate, toxin, metabolism stimulator, etc. is added to the ports 230 of cartridge 110 before beginning an analysis using a pipette or other means. The compound typically will be an aqueous solution of a known concentration. In preferred embodiments, it is held within each port despite the presence of a small outlet at its bottom by surface tension. The dimensions of the port inhibit leakage from the bottom and from the top end (forming a meniscus that prevents leakage if the apparatus is turned on its side or upside down). The test compound may be released by, e.g., the application of pressurized air.

It may be desirable to operate the apparatus with test liquids that are difficult to contain using capillary force due to their relatively low viscosity or electrostatic properties. In this case, a frangible membrane or a fragile material, such as wax may be attached to cover the hole in the bottom of the port 230, such that an extrinsic force can breach the membrane to eject the liquid at a desired time.

In the depicted embodiment, the submersible sleeve 240 is disposed between first and second ports 230. Sensors 250, e.g., fluorophores, are disposed on surface 325 at the lower end of the sleeve. The submersible sleeve 240 is configured to receive the sensor probe 170.

An array of integrated sensor sleeves and compound storage and delivery ports may be fabricated as a single assembly using a low cost fabrication process such as injection molding so that the cartridge may be disposed of after use.

Referring to FIG. 5, the well 220 is formed of, e.g., molded plastic, such as polystyrene or polypropylene. In use, cell media 500 and live cells 510 are disposed in the well 220. Cells 510 may or may not adhere to a bottom surface 520 of the well, and the bottom surface may be treated or coated to encourage adherence. Alternatively, cells may be suspended within the media.

Referring to FIGS. 6a and 6b, in use, when the parts are assembled, they allow simultaneous sensing of constituents in the cell media in plural wells simultaneously, and delivery of test compound from the ports.

As illustrated, the fixed probe structure and drug loaded cartridge are assembled such that the outer tubing holding the fiber optic bundle is disposed within the sleeve of the cartridge, and the assembly is reciprocated from an up position, where the probe tip and sensors are disposed in the cell medium, to a lower, data gathering position, one that reduces the volume of media about the cells so as to improve the ability of the sensor to detect changes in the concentration of an analyte in the media about the cells (see US 2005/0054028). In the preferred embodiment, the sensors 250 disposed on the lower surface 325 of the sensor sleeve 240 remains submerged during mixing, equilibrating, and measurement steps. One or more constituents within the media secreted from or absorbed by the cells may by analyzed. In a first lowered position (FIG. 6a), a fluid, such as a drug sample, is delivered from one of the ports 230 to the cell medium, in this embodiment impelled by air pressure communicated through air channels 310. As noted above, the drug may be released through a small hole disposed at a bottom portion of the port 230.

After the fluid is dispensed into the media, the sensor sleeve 240 may be raised and lowered one or more times while remaining submerged in the media to mix the fluid with the media. The sensors 250 may remain disposed within the media during the dispensing and mixing steps, thereby reducing stabilization periods.

After the test fluid is dispensed and mixed with the media, the sensors 250 and sensor sleeve 240 are lowered to a second lower position in the well 220. A bottom portion of the well 220 may include a seating surface for the sensor sleeve 240, e.g., an internal step defining a step plane above a bottom plane of the well 220, the step plane and bottom plane being parallel planes. In a microwell microplate, the height of the step plane may generally be less than about 1 mm above the bottom plane and typically less than about 50 μm to 200 μm above the bottom plane. Alternatively, a flat bottomed well or other well configuration may be used, and the fluorophore probes may disposed on surface 255 within a recess formed by a wall extending slightly beyond the surface as disclosed above. In either case, in this embodiment a small volume subchamber is formed about cells when the assembly is disposed in a down position. Relatively small changes in the concentration of the constituent then can be detected by the fluorophore probes, as the measurement is taken within the confines of a much smaller volume of medium. This subvolume is maintained for a short time period to make a measurement, and the assembly is moved upwardly, permitting the cells to be exposed to the full well volume of its medium.

In an alternative embodiment, the test fluid from the port may be delivered to the media when the sensor sleeve in the partially raised, but still submerged position.

During or after the delivery of the test fluid to the well, the constituent in the medium may be analyzed to determine any changes, and the measurements can be repeated with or without intermediate addition of test compounds. Any number of constituents of the media may be analyzed, including dissolved gasses, ions, proteins, metabolic substrates, salts, and minerals. These constituents may be consumed by the cells (such as O₂), or may be produced by the cells either as a byproduct (such as CO₂ and NH₃) or as a secreted factor (such as insulin, cytokines, chemokines, hormones, or antibodies). Ions such as H⁺, Na⁺, K⁺, and Ca⁺⁺ secreted or extracted by cells in various cellular metabolism processes may also be analyzed. Substrates either consumed or produced by cells such as glucose, fatty acid, amino acids, glutamine, glycogen, and pyruvate may be analyzed. Specialized media may be used to improve the sensitivity of the measurement. For example, a change in pH resulting from extracellular acidification can be increased by using a media with reduced buffer capacity, such as bicarbonate-free media.

The method may be used to measure any number of attributes of cells and cellular function. For example, cell vitality and metabolic rate may be determined from measurements of oxygen consumption rate, extracellular acidification rate, or other metabolic analyte fluxes. By comparison of one or more analyte flux rates to a known rate per cell, cell number may be determined and therefore growth rates can be monitored.

The introduction of an environment altering constituent such as a chemical, dissolved gas, or nutrient may be applied to either the full volume of the well or alternatively to only the reduced volume of the well. In the latter embodiment, the volume of media surrounding the cells is first reduced, the constituents of the media are measured, and the volume is restored to its original value. The volume is then again reduced and the environment immediately surrounding the cells within only the reduced volume is then altered, by the addition of a constituent from one of the four corresponding ports. This may be accomplished by discharging the constituent from a port proximate the sensors or the bottom of the sleeve, for example. One or more measurements in the reduced volume are made in the presence of the constituent. After this measurement cycle, the media within the reduced volume may be exchanged one or more times to flush out the constituent before exposing cells once again to the full original volume. This approach may provide a benefit of reducing the volume of compound required. It may also provide the possibility of studying isolated effects without contaminating the entire volume, thereby, in effect, simulating a flow system in microplate format.

In preferred embodiments, as illustrated in the drawing, a plurality of sensors are inserted and disposed simultaneously or sequentially in a corresponding plurality of wells in the multiwell plate, and constituents related to respective cell cultures in respective wells are analyzed. The respective constituents may include the same constituent. Respective test fluids may be delivered to the respective wells while the respective sensors remain in equilibrium with, preferably remain disposed within the media in respective wells. It is possible to maintain equilibrium with many sensors, particularly fluorophore sensors, while the sensor body is removed from the media for a short time, e.g., if the probe remains wetted, permitting maintenance of equilibrium while adding test fluid. In one embodiment, the respective test fluids may be the same test fluid. The respective constituents related to respective cells within media in respective wells may be analyzed to determine any respective changes therein. These delivery and analysis steps may be repeated. In another embodiment, the delivery step is repeated with a different test fluid.

In some instances, the delivery and analysis may be repeated after a time period. More particularly, sequential measurements of a single group of cells may be made at predetermined time intervals to analyze the effect of a compound addition temporally, for example to examine the effect of exposure to a drug, chemical, or toxin. In this method, the volume of media surrounding the cells is first reduced, the constituents of the media are measured, and the volume is restored to its original value. The environment surrounding the cells is then altered, such as by adding one or more predetermined concentrations of a ligand that activates a transmembrane receptor, changing the dissolved oxygen level, or adding a nutrient. One or more additional measurement cycles then are performed using the temporarily reduced volume method, to analyze the effect of the altered extracellular environment.

Equilibration between the sensor and the media may be maintained during the delivery step. Thermal equilibrium may be substantially maintained between the test fluid and media during the delivery.

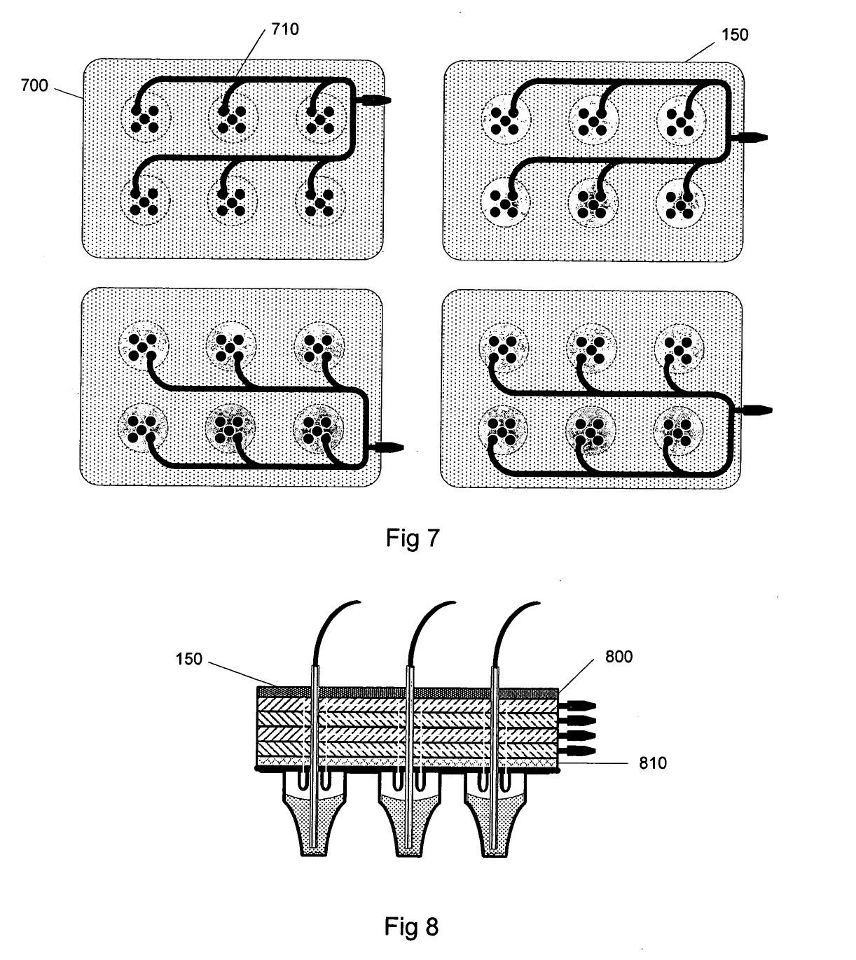

Referring to FIGS. 7 and 8, the currently preferred form of the multiplexer 150 is shown. It comprises a laminated assembly of multiple layers 700 of planar polymeric sheet material containing machined channels 710 for gas flow, sandwiched between a cover sheet 800 and cartridge facing gasket 810. One such arrangement uses four layers, e.g., four machined blocks placed in different orientations, to create a pneumatic multiplexer enabling the dispensing of fluid from any one of four ports disposed in each region of the cartridge. The multiplexer enables the delivery of gas from a single gas inlet to multiple outlets. In use, the multiplexer is disposed between a pressure source and the cartridge, with the multiplexer adapted to be in fluidic communication with a plurality of ports formed in the cartridge. The multiplexer may be selectively in fluidic communication with an exclusive set of ports formed in the cartridge.

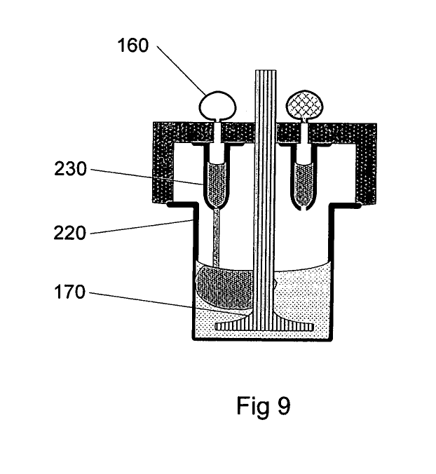

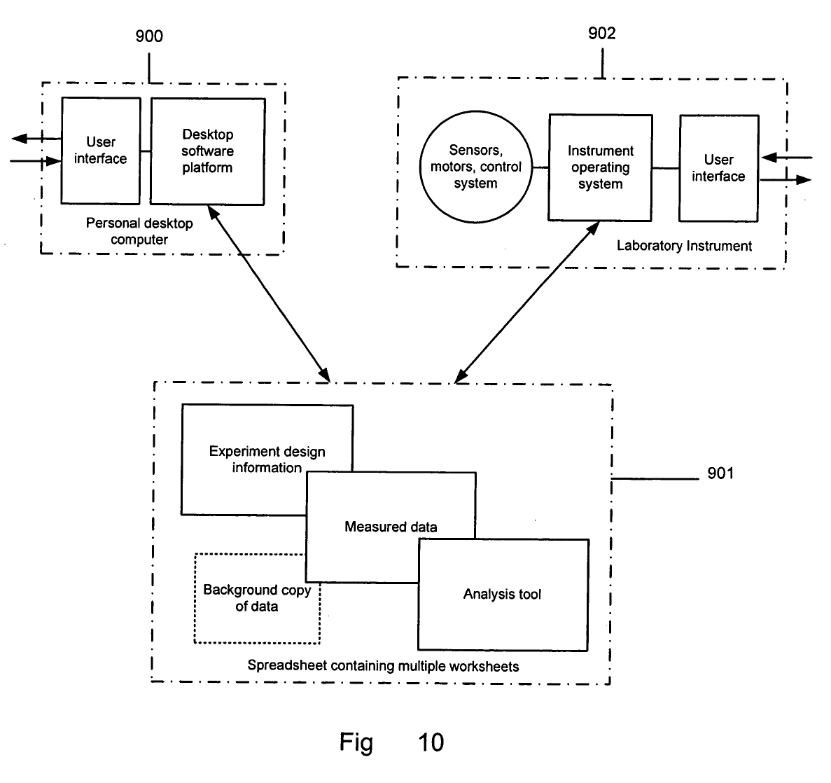

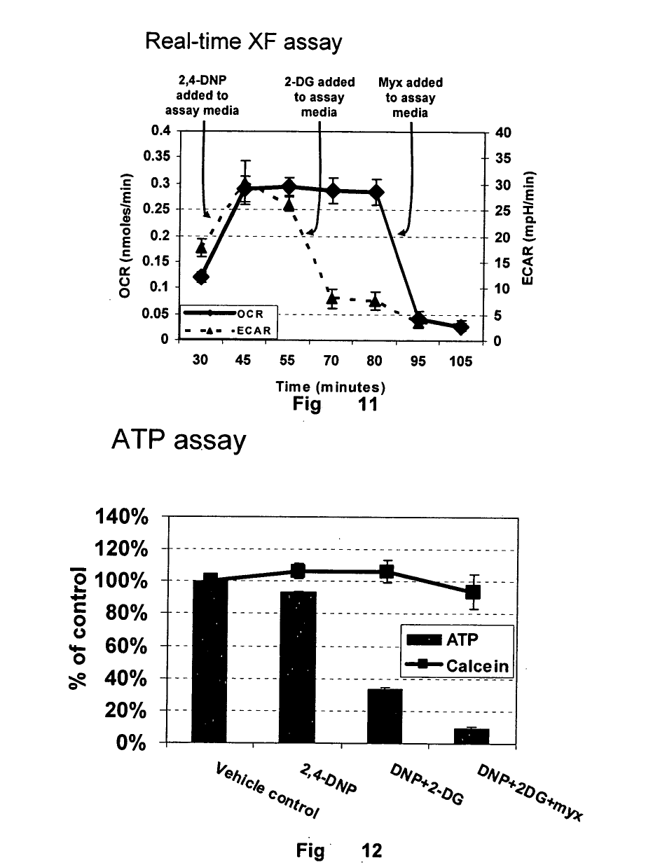

Referring to FIG. 9, the sensor probe 170 is depicted submerged in the liquid media contained within a microplate well 220. The drug delivery apparatus is shown activated using gas pressure from the pressurized gas supply 160 to deliver a drug from the port 230 to the media.

FIG. 10 schematically illustrate one embodiment of the apparatus useful in the practice of this invention realized as an instrument and software for analyzing cells undergoing various experimental processes using any of the techniques described above. A key element of the apparatus is a data file shared by instrument operating system running on the embedded instrument computer, and desktop software running on a user's personal desktop computer.

As illustrated in FIG. 10, desktop software 900 contains a user interface that allows a user to enter experiment design information into data file 901. Experiment design information may include the type of cells, number of cells, type of drug, and concentration of drug contained in each microplate well, the required measurement time, media mixing time, the analyte to be assayed, or other data that define attributes of the experiment to be run by the instrument.

Instrument operating system software 902 both receives experiment design information from, and stores experiment results to, data file 901. Operating system software 902 also contains a user interface for viewing and modification of experiment design information and for viewing of experiment results.

The instrument operating system software provides actuation and control of motors, heaters and other devices based on the settings provided in the data file. During each measurement cycle, measured data may be displayed on the user interface and concurrently added to the data file. At the end of a complete experiment, the data file, containing experiment definition data, and measured sensor data, may be stored and transmitted to the user's desktop computer for analysis. The user may a third-party analysis software package that draws data from the data file. Examples of suitable third-party analysis software include MICROSOFT EXCEL (Microsoft Corp), JMP (SAS Corp), and SIGMA PLOT (Systat Corp).

In a preferred embodiment, data file 901 is in the form of a spreadsheet.

B. Fundamental Biology and Biochemistry on which the Methods of the Invention are Based and Description of Protocols

As is apparent from the foregoing, insight into a cell's choice of nutrient and metabolic pathway can be gained from measurements of the flux rates of nutrients, gasses, ions, and other analytes between the cell, tissue or isolated cellular organelle and the external aqueous environment (media). Glucose, for example, can be used to produce ATP and biosynthetic precursors through the rapid process of glycolysis as follows:
Glucose to lactic acid and ATP: C₆H₁₂O₆→2C₃H₆O₃+2ATP
Lactic Acid to lactate and protons: 2C₃H₆O₃→2C₃H₅O₃⁻+2H⁺

Glycolysis consumes no oxygen, and produces a significant amount of lactate and free protons as a byproduct, which acidify the surrounding media.

When oxygen is available, glucose can be converted through the ATP-rich, but slower aerobic process through the overall metabolic reaction shown below:
C₆H₁₂O₆+6O₂→6CO₂+6H₂O+36ATP

The carbon dioxide that is produced through this process is converted to carbonic acid by carbonic anhydrase which can then dissociate to release bicarbonate and protons:
CO₂+H₂O→H₂CO₃>HCO₃⁻+H⁺

With all other factors constrained, a measurement of extracellular flux rates of a cell entirely dependent on aerobic respiration of glucose differs from a measurement of a cell entirely dependent on anaerobic metabolism of glucose in that the flux rate of oxygen is higher, and the flux rates of glucose, lactic acid and protons are lower. Thus, a measurement and comparison of two or more appropriately chosen extracellular fluxes can provide an indication of the glucose conversion pathway in use within the cell.

Further elucidation of metabolic pathways can be obtained with the use of drugs that modulate specific mechanisms within the primary metabolic processes. In this method, baseline extracellular flux rate measurements are made, then metabolic modulators are added to the assay medium, optionally at various concentrations, and flux rate measurements are repeated at various time intervals post exposure. Metabolic modulators include, but are not limited to, glycolysis inhibitors, mitochondrial uncouplers and inhibitors, and pentose cycle inhibitors. Metabolic modulators that affect the trans-plasma membrane electron pathway (tPMEP) such as NADH and capsaicin may also be used. Dose-response curves are then generated for each rate, and the sensitivity and degree of inhibition are determined from the dose response curve.

This method of analysis can provide insight into the preference and/or dependency of a cell type in a particular biological state on either glycolytic energy production or mitochondrial energy production, which may be caused, for example, by a defect in the glycolytic or mitochondrial respiration pathways. More generally, this method can provide an indication of both the acute and chronic effect of a drug on differentiation, proliferation, apoptosis, necrosis, senescence, autophagy, oncogenic phenotype and malignancy. Specific patterns of metabolic changes may, in fact, be used as markers of the onset of apoptosis and or necrosis or the process of sequestering organelles or long-lived proteins in a double-membrane vesicle inside the cell, where the contents are subsequently delivered to the lysosome for degradation (autophagy).

The effect of new drug candidates or gene therapies can be analyzed by comparing basal flux rates of cells to flux rates following exposure. The use of metabolic modulators for further exploration as described above can be applied to cells post treatment with a candidate drug or gene therapy.

Further information can be obtained by comparing the effect of a drug, genetic modification or environmental change in two or more cell types, where the types have significantly different metabolic mechanisms. For example, experiments may be performed to compare the response or characteristics of HeLa cells to their mitochondrial-deficient HeLa rho0 counterparts.

Another approach is to determine the effect of a drug or combinations of drugs, agonists, antagonists, genetic modifications or environmental changes in two or more tissues or organelles. For example, experiments may be performed to compare the response of muscle tissue biopsies isolated from different individuals or from the same individual after various exposure to different agents.

One specific area of research that can benefit from such real time, sensitive cellular metabolism data is the field of metabolic disease drug discovery. One particularly interesting drug target for therapies to treat obesity and diabetes is the process of fatty acid oxidation, primarily within muscle and fat cells and tissues.

The most common methods for measuring fatty acid oxidation require incubating cells in media containing a radiolabeled fatty acid such as palmitate or oleate, then capturing and measuring the radiolabeled CO₂ or H₂O that are byproducts of the FA metabolism. Radioactive assays are typically slow, laborious and require the handling and disposal of costly radioactive materials.

Certain nutrients, including fatty acids, can be converted to ATP and/or reducing equivalents to produce heat only through an aerobic process such as the following:
C₁₆H₃₃O₂+23O₂→16CO₂+16H₂O+129ATP

In comparison with glucose oxidation, fatty acid oxidation produces relatively fewer protons (approximately 30% fewer) as a byproduct than glucose oxidation due to the lack of production of carbonic acid as it enters into oxidative metabolism as compared to the initiation of glucose oxidation that produces a molecule of carbonic acid when pyruvate is converted into Acetyl CoA+CO₂.

As a result, the measurement and comparison of two or more extracellular fluxes, such as oxygen consumption rate (OCR), proton/extracellular acidification rate (ECAR), CO₂ production rate (CPR) and/or lactate or glucose fluxes, can provide discrimination between fatty acid and glucose oxidation within a cell. Quantitative measures of the metabolic rate per cell for various substrates and pathways can be obtained if the flux measurements are normalized to cell number, vitality, mg protein or ATP yield.

One protocol useful in the search for drugs modulation fatty acid metabolism is to simultaneously or serially add a fatty acid and a candidate drug to a test well, preferably containing primary animal cells, and then to observe the effect on fatty acid metabolism induced by the candidate drug. There are many possible permutations on such experiments. Thus, one can develop dose response curves, compare different candidate drugs in different wells simultaneously, assess the relative use of aerobic versus anaerobic metabolic pathways in the presence of different fatty acid substrates, etc.

Since it may not always be possible to constrain the many variables that affect metabolic rates and pathways, compounds that are known to augment or inhibit specific metabolic pathways, including oxamate, phloretin, and myxothiazol may be used in conjunction with extracellular rate measurements to identify specific pathways and/or reduce variability as shown in the examples below.

Another area of research in which cellular metabolic data may be useful is the area of screening new or known drug compounds, drug candidate compounds, or genetic and immuno-therapies for new or off-target and/or adverse (toxic) effects. A thorough search for off-target, toxic, or other unexpected responses of cells to drugs or genetic alternations is an expensive and time consuming effort. For this purpose, an assay that is responsive to multiple cellular functions is attractive as a screening tool, since it can potentially replace many tests of the numerous specific functions of a cell.

Alternatively, one may administer a substance to an animal, and sample cells from the animal once or multiple times during a treatment regime. The sample cells are tested using the protocols disclosed herein.

In one aspect, to conduct an assay, one first incubates the cells under investigation in a medium disposed in at least one of a plurality of wells in a multi-well plate. Typically, multiple experiments are run in parallel. Next, one adds to the medium so as to bring into contact with the cells a substance potentially capable of altering cellular metabolism. This can be done as described above using the fluid distribution system of the CellDoctor™ device. Next; one measures in the medium in a well, typically multiple wells simultaneously, the rate of change in concentration of both an extracellular solute which is a component of cellular aerobic metabolism and an extracellular solute which is a component of cellular anaerobic metabolism. This takes multiple individual measurements over a time interval so as to generate data indicative of the change in concentration in the medium, as opposed to data indicative of the concentration at any given time.

Any type of animal cell, tissue or organelle may be used. The cells under analysis may be, for example, primary animal cells, such as cells growing on a surface in a well, neoplastic cells, or cells disposed in suspension. The tissues under analysis may be, for example, freshly isolated or thawed, cryopreserved tissue sections such as muscle, adipose, liver and brain. The organelle under analysis may be, for example, isolated mitochondria.

Any fluid which is soluble in the media (or a substance which can affect cells in non soluble forms) can be used. The substance may be a drug or drug candidate, a toxin, a ligand known or suspected to bind to a cell surface receptor, a nutrient, a cytokine, a growth factor, a chemokine, a metabolism inhibitor or stimulator, or any biomolecule such as an antibody—essentially any soluble molecule potentially capable of perturbing the biological state of the cells. Preferably, as discussed above multiple measurements (at least two) are conducted substantially simultaneously in a well, e.g., by collecting data through the various fluorescent probes.

Alternatively one can measure a flux prior to addition of the substance, or in the medium of a cell culture in a well separate from the cells under analysis, and values from the separate measurements may be compared to infer knowledge concerning the biology of the cell and/or the biological properties of the substance. In yet another alternative, one may measure in the medium of a cell culture in a well separate from the cells under analysis, and treated differently from the cells under analysis, either or both the rate of change in concentration of extracellular solutes which are respectively components of cellular aerobic and anaerobic metabolism. Thus, data from multiple wells treated with different concentrations of the same substance can be used to construct a novel type of dose response curve. Alternatively, the same data may be obtained by making multiple serial additions of the substance to increase its concentration in the media in a single well serially, and making measurements after each addition. In some cases, by comparing the measurements of the rates of change in the separate cell cultures one can directly compare potency of different drugs. Also, the method may be practiced by measuring in the cell medium the rates of change in concentration at different times to obtain a temporal profile of the effect of the substance on said cells.

The measured component of cellular aerobic metabolism is preferably extracellular oxygen, and the measurement is oxygen consumption rate (OCR). The measured component of cellular anaerobic metabolism is preferably extracellular proton concentration (extracellular acidification rate −ECAR), carbon dioxide production rate (CPR), lactic acid production rate, or lactate production rate. Other molecules absorbed or secreted by animal cells and related to metabolic activities also may be exploited.

In yet another embodiment, the invention provides a method for analysis of cell culture “quality” or vitality that is a measure of how close a cell culture is to healthy homeostatic state, as opposed to having some portion of the cells dead or in the process of dying, or of greatly reduced metabolic capacity, due to some stress or challenge. This is done by measuring in a cell medium the rate of change in concentration of both an extracellular solute which is a component of cellular aerobic metabolism and an extracellular solute which is a component of cellular anaerobic metabolism both in a basal and challenged state (such as by exposure to an mitochondrial uncoupler such as 2,4-DNP), followed by comparing the measured rates of change to a standard informative of known cell culture respiration rates. This procedure serves to assess the basal respiratory rate and capacity of the culture and can be a sensitive measure of cell vitality, quality, and health. In one form, this cell quality measurement method may involve comparing the measured rates of change to a standard indicative of rates measured in a culture comprising a known number of healthy cells of the same cell type or of a cell type comparable to the cells under quality assessment. In another form, one may seed cells at a predetermined density in a test well (or go through a cell counting procedure) prior to making the measurement thereby to enable direct comparison of the measured rates of change to a standard, e.g., on a per cell, or per 10⁴ cell basis. In another form, the cells can be conditioned to generate an appropriate increase in fatty acid oxidation by optimizing their culture media components and or conditions, such as but not limited to, glucose, carnitine, serum, glutamine, electrolytes, % O₂.

These methods do much more than take a measurement indicative of whether the cells in a culture are alive, or what fraction of the cells are alive, as it can measure metabolic rate; measure relative contribution of aerobic (oxidative phosphorylation) versus anaerobic (glycolysis) processes which generate ATP. Furthermore, the quality assessment is non destructive, and therefore the planned experiment on the cells can be conducted after assessing cell vitality and quality.

In a related aspect, the invention permits the scientist to obtain data indicative of respiratory (or metabolic) capacity of a cell culture without cell counting. This is done by measuring a basal metabolic rate or rates (i.e., rates of change of OCR, ECAR etc.), before the addition of any metabolism altering substance, followed by adding to the culture a drug that increases metabolism (uncouplers), and then repeating the measurement. The change is indicative directly of metabolic capacity, and the ratio can be used as such a measure independent of the actual amounts of cells in the test well in which the measurements were made. This eliminates the need for cell count to normalize data, and can be particularly valuable when cell number is different in various wells or when cells proliferate during the experiment (particularly cancer cells).

Another way to normalize the metabolic data is to measure the basal metabolic rate or rates (i.e., rates of change of OCR, ECAR etc.), before the addition of any substance that alters the metabolism and then remove the cells or an aliquot of cells or add directly to the cells a reagent, such as a dye specific for mitochondria, to express the metabolic changes based upon the total amount of mitochondria.

In certain embodiments, methods of the invention include profiling the metabolic function of living cells by comparing measurements of the extracellular flux rate of at least two analytes. These methods may be used to assess aerobic versus anaerobic metabolism, including the following exemplary situations: where at least one analyte is sensitive to aerobic metabolism and at least one second analyte is sensitive to anaerobic metabolism; where one analyte is sensitive to either aerobic or anaerobic metabolism alone and a second analyte is insensitive to both; where an analyte sensitive to aerobic metabolism is O₂, CO₂, and the like; where an analyte sensitive to anaerobic metabolism is proton flux (pH change), lactate, and the like; or where two analytes, one sensitive to aerobic and another sensitive to anaerobic metabolism can be used to calculate the amount of ATP generated per unit of time and the percent uncoupled metabolism per unit time.

The methods may be used to measure a change in cellular metabolic function induced by exposure to a toxin such as one that induces necrosis or apoptosis; or a toxin that arrests proliferation, differentiation, or impairs nutrient transport, conversion, or mitochondrial function. Such a change can be detected and is reflected in a change in the rate of change of concentration of selected extracellular solutes. Alternatively, or in addition, the methods may detect and quantify changes in cellular metabolic function induced by exposure to a drugs or drug candidates, genetic modifications that induce necrosis, apoptosis, or metabolic impairment; or environmental challenges that induce necrosis, apoptosis, or metabolic impairment.

In one important class of activities, the methods of the invention permit determination of the effect of candidate drug compounds on anaerobic glycolysis in cancer cells by measuring the relative flux rates of analytes that are sensitive to glycolysis versus insensitive to glycolysis or sensitive to oxidative phosphorylation. Cells may be treated, measured, and compared to untreated cells. Treatment may include drug exposure, genetic modification (RNAi and the like), and environmental changes (pH, temperature, radiation and the like). The baseline may be measured, the cells treated, and the measurement repeated one or many times.

In another important class of activities, the methods of the invention permit non-invasive assessment of the magnitude of fatty acid oxidation (FAO) within living cells by measuring the relative flux rates of analytes that are sensitive to versus insensitive to FAO, or sensitive to FAO versus sensitive to metabolism of other nutrients including glucose and amino acids. The methods may be used with analytes sensitive to FAO, i.e., O₂. Analytes insensitive to FAO include protons and lactate.

The following examples demonstrate that extracellular flux rate measurements can predict both acute and chronic cytotoxic effects of drugs in vitro, measure metabolic properties of a number of cell types, and can be used to profile the effects of various drugs. A method for measuring fatty acid oxidation by cells, and a demonstration of cell quality assessment in accordance with the invention are also are described.

### EXAMPLES

### Example 1

### Real-Time Measurement of Changes in Cellular Energetic Pathways in LNCaP Cells in Response to Metabolic Modulators

Cancer cells were exposed to a series of drugs that impact metabolic function in order to determine the ability of the extracellular flux (XF) assay to interrogate changes in metabolic function. Prostate cell line LNCaP was obtained from the ATCC (Manassas, Va.). LNCaP cells were maintained in modified RPMI 1640 media supplemented with 10% fetal bovine serum (FBS) and 100 μg/ml penicillin-Streptomycin. A highly invasive and metastative, in vivo-derivative of LNCaP, C₄₋₂ cells were maintained in T medium. 2,4-DNP, 2-deoxyglucose, myxothiazol and Calcein AM were prepared according to the manufacturer's instructions. Metabolic flux rate measurements were performed using a prototype Seahorse XF instrument as described above. This instrument was configured to measure the oxygen consumption rate (OCR) and extracellular acidification rate (ECAR), corresponding to proton flux, of cells that are adherent to the bottom of 24 well microplates. The first step in a measurement sequence was the exchange of cell media to a type containing nearly no bicarbonate or other pH buffer in order to maximize the pH change caused by cellular proton flux.

Next, measurement probes fitted with an optical oxygen and pH sensor were placed in each well, and were raised and lowered to mix the cell media for approximately five minutes. Third, each probe was lowered into the cell media to stop at a precise point above the cells such that approximately 25 μL of media was sequestered in a way that impeded the diffusion of oxygen molecules and protons to the larger volume of media in the well. Fourth, a series of optical measurements of the two sensors on the probe bottom (in contact with the small volume of media above the cells) were made at intervals of 8 seconds and for a period of five minutes. Typically, the dissolved oxygen level decreased by approximately 10% and the pH decreased by approximately 0.1 units. A simple linear fit to the rate of change of oxygen and pH were used to determine the OCR and ECAR for each well.

Next, the probes were elevated and then reciprocated to re-mix the depleted media with the larger residual volume in the well. Typically, both the dissolved oxygen level and pH of the mixed media returned to the starting values. This basal rate measurement was typically repeated.

In cases where drug addition was to be used, 100 μL of a 10× compound solution was added to the cell media while the sensor probe was elevated but resident in the cell media. A mix cycle and measurement cycle were then performed (as previously described) and both were repeated again. In some cases, second and third drug compounds were added with mix and measure cycles for each. Baseline metabolic rates were reported in nmol/min for OCR and mpH/min for ECAR. At the completion of each assay, cells were suspended from the well bottoms using Trypsin, and a viable cell number count was obtained using an automated Trypan Blue dye exclusion type cell counter.

ATP assays were performed in parallel with metabolic flux measurements. Cells were seeded in opaque 96-well tissue culture microplates at indicated cell density per well 24 hours prior to compound treatment. Cell Titer-Glo™ luminescent ATP assays were performed at the indicated treatment time using a FLUOstar Optima™ plate reader.

For Calcein AM assays, cells were seeded in black 96-well tissue culture microplates at a density of 10,000 cells per well. Calcein AM (2 mM) stain assays were then performed per the manufacturer's instructions.

Mitochondrial uncoupler 2,4-DNP (100 μM), glycolysis inhibitor 2-deoxyglucose (50 mM) and mitochondrial complex III inhibitor, myxothiazol (0.1 μM) were injected sequentially into wells containing LNCap cells. Oxygen consumption rate (OCR) and extracellular acidification rate (ECAR) were measured before and after compound injection.

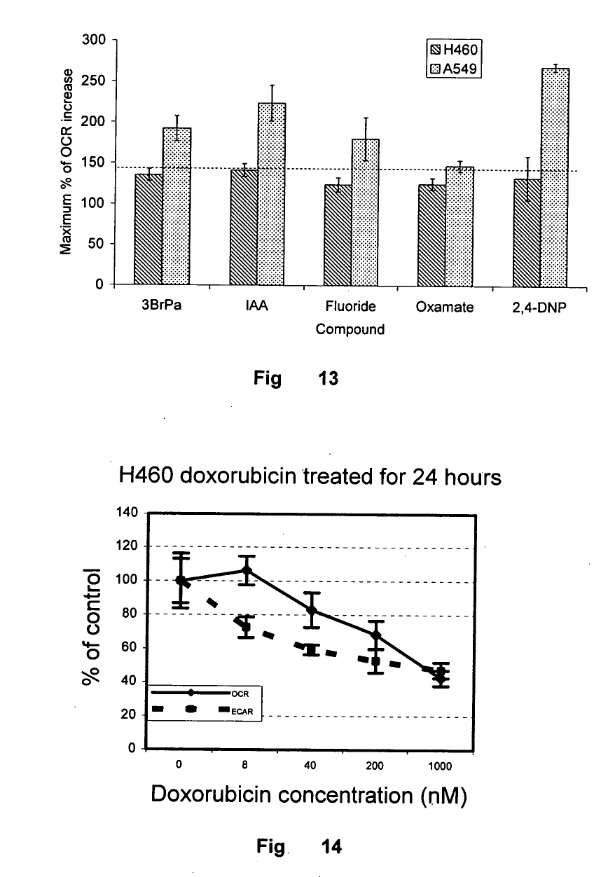

As shown in FIG. 11, exposure to 2,4 DNP caused a dramatic increase in both OCR and ECAR within ten minutes of compound administration. The elevated ECAR was then inhibited by the hexokinase inhibitor, 2-Deoxyglucose that was injected 25 minutes after 2,4-DNP administration, while OCR remained high. The final injection of myxothiazol at 25 minutes after oxamate administration diminished cellular oxygen consumption rate.

Cells remained viable under all conditions as assayed by Calcein AM stain, and the ATP levels for each treatment corresponded to the relative changes in metabolic rates (see FIG. 12).

### Example 2

### H460 Cells Exhibit Attenuated Mitochondrial Respiratory Capacity as Compared to A549 Cells

Experiments were performed as disclosed above to demonstrate and confirm the increased glycolytic capacity and more aberrant mitochondrial respiration in a highly metastatic cancer cell line.

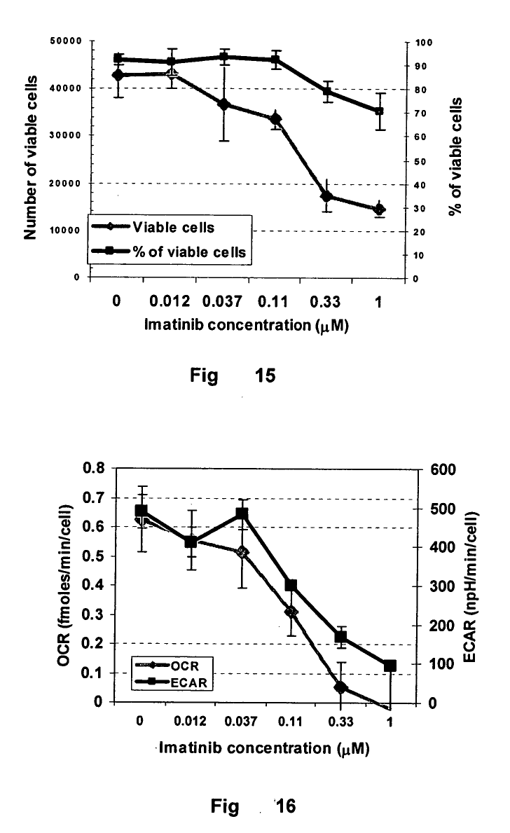

As shown in FIG. 13, a maximal increase of 150% in OCR over baseline was observed in H460 cells in response to glycolysis inhibitors (A), oxamate, 3-bromopyruvic acid, iodoacetate and fluoride. Since the maximum OCR increase following treatment with the mitochondrial uncoupler 2,4-DNP was also 150%, this suggests that lower mitochondrial respiratory capacity is an intrinsic characteristic of H460 cells (B). In comparison, maximal 225% and 250% responses were observed in A549 cells following glycolysis inhibitors or 2,4-DNP respectively.

### Example 3

### Doxorubicin Mediated Cytotoxicity in H460 Cells

XF assays were used to profile the dose response of the chemotherapeutic compound Doxorubicin. H460 cancer cells were exposed to various doses of Doxorubicin for 72 hours, then profiled using XF assays as described above. As shown in FIG. 14, a biphasic OCR response was measured in which oxygen consumption was enhanced at doses below 10 nM and inhibited at doses exceeding 10 nM, suggesting stimulation of aerobic metabolism to support increased ATP demands of the cell at doses below the highly cytotoxic concentrations typically used for therapy.

### Example 4

### Altered Energy Metabolism in Human K562 Cells 48 Hours after Incubation with Imatinib (Gleevec®)

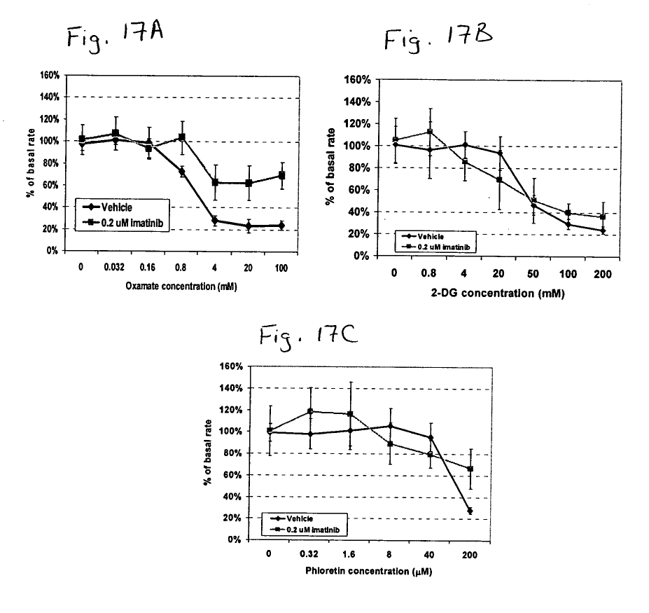

An experiment was performed to demonstrate the relationship between cancer cellular bioenergetics and established cancer drugs or agents that modulate oncogenic pathways. K562 cancer cells were seeded at 6,000 cells per well and incubated in growth medium containing DMSO and Imatinib mesylate (Gleevec®) at concentrations of 0.1%, 0.012, 0.037, 0.11, 0.33, and 1 μM for 48 hours. The number of viable cells and percent of viable cells were determined by automated Trypan blue counting (FIG. 15).

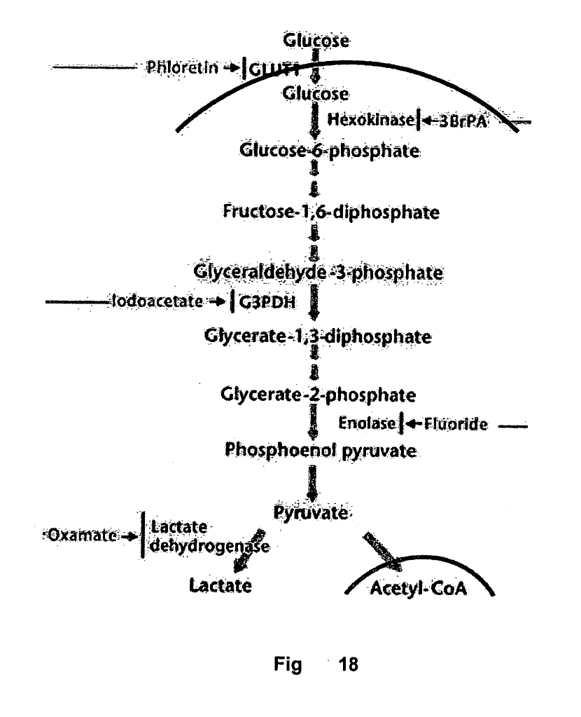

An equal number (80,000/well) of viable cells, after 48 hours incubation in growth media containing DMSO at concentrations of 0.1%, 0.012, 0.037, 0.11, 0.33 and 1 μM of Imatinib, respectively, were seeded onto a Cell-Tak coated 24-well plate. The cells were allowed to attach themselves to the surface of the wells for 1 hour prior to XF measurement. Cells were counted after XF-assay and OCR and ECAR per cell were calculated. Metabolic flux rates and cell vitality data are shown in FIG. 16.

These data show that Imatinib reduced the glycolysis rate of a BCR-ABL-positive and Imatinib-sensitive cell line, K562, in a dose dependent manner. Furthermore, bioenergetic profiling of Imatinib treated and untreated cells revealed changes in sensitivity and degree of inhibition by glycolysis blockers.

### Example 5

### Cell Bioenergetic Profiling of K562 Cells Treated with Imatinib

K562 cells at 100,000/ml were incubated in 0.2 μM Imatinib for 48 hours before they were counted and seeded onto Cell-tak coated 24-well plate. Subsequently, the cells were profiled for their response to increased concentration of oxamate, 2-DeoxyGlucose and phloretin in the XF assay.

As shown in FIG. 17A, FIG. 17B and FIG. 17C, Imatinib pre-treatment reduced the sensitivity and extent of inhibition of ECAR resulting from exposure to LDH inhibitor oxamate in K562 cells, suggesting the down regulation of LDH. Sensitivity to and extent of inhibition by the hexokinase inhibitor 2-deoxyglucose were slightly lowered in Imatinib treated K562 cells. The degree of inhibition by GLUT1 inhibitor was significantly reduced in Imatinib treated K562 cells. This observation is useful for developing a better understanding of the mechanism of the action of the drug especially with regards to its affect on metabolism.

### Example 6

### Cell Bioenergetic Profiling of H460 and A549 Cells

Experiments were run to demonstrate that acute oxygen consumption rate (OCR) and extracellular acidification rate (ECAR) change in H460 and A549 cells in response to inhibitors of glycolysis indicated a concomitant increase in OCR as ECAR decreases. The highly metastatic H460 cells showed a greater sensitivity to lactate dehydrogenase inhibitor oxamate.

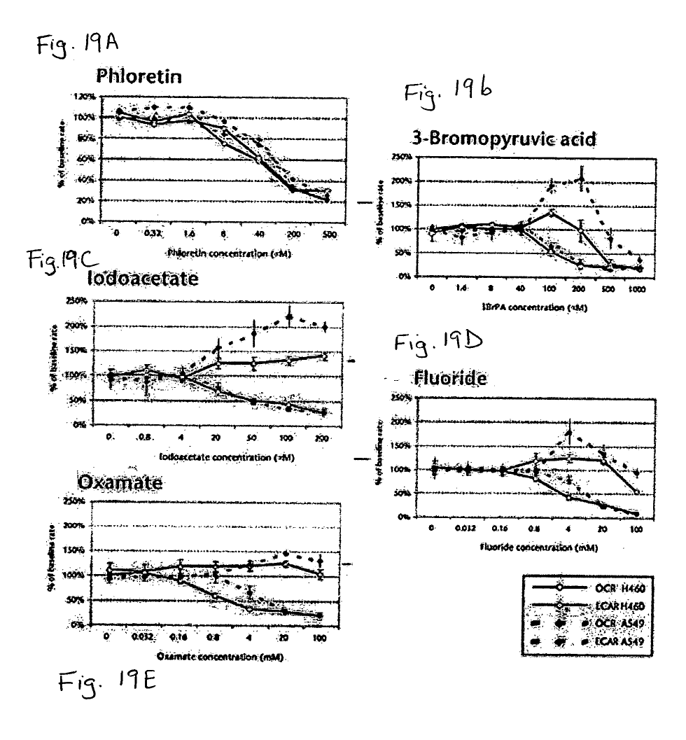

Thus, the effect of various compounds that modulate specific steps of the metabolic pathway (as shown in FIG. 18) were profiled. Percent change in OCR and ECAR relative to baseline, in response to compound treatments are shown in FIGS. 19A-19E. Note: The GLUT1 inhibitor phloretin also inhibited mitochondrial respiration resulting in decreased OCR and ECAR. This observation is valuable for exploring the differences between highly metastatic and less metastatic tumor cells to support development of therapies for particularly aggressive cancer types.

### Example 7

### HIF-1 Mediated Recapitulation of the Warburg Effect in LNCaP Cells

An experiment was performed to demonstrate directly small molecules that either increase or decrease HIF-1α expression which is known to mediate the Warburg effect (anaerobic glycolysis) and its reversal, respectively.

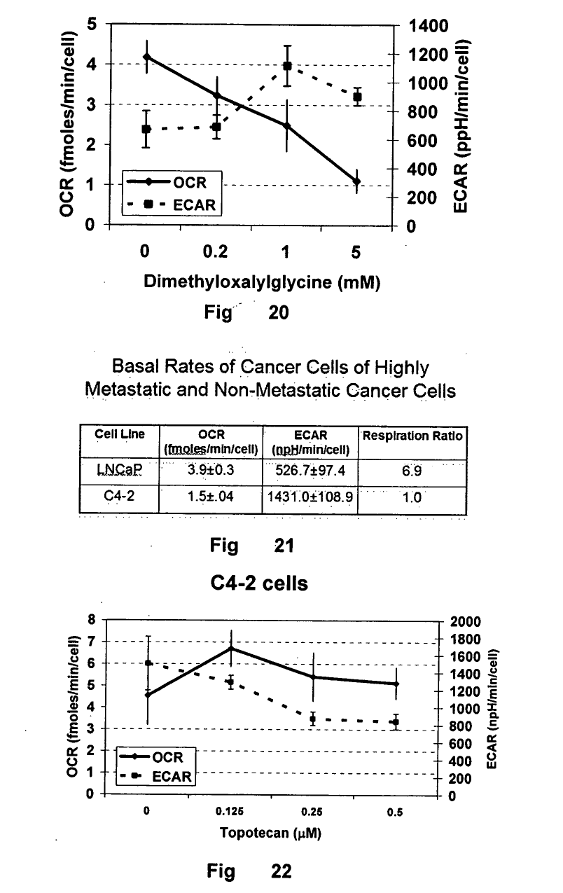

LNCaP cells were exposed to increasing concentrations of dimethyloxalylglycine (DMOG) for 24 hours. DMOG, a prolyl hydroxylase inhibitor (which increases the half life of HIF-1α by preventing VHL-dependent HIF-1α destabilization) stimulated the cellular rate of glycolysis and simultaneously suppressed the cellular rate of mitochondria respiration (FIG. 20). This suggests that HIF-1α down regulated expression of mitochondria respiration genes in addition to its reported activity of upregulating glycolytic enzyme expression. OCR and ECAR were normalized to rate per cell.

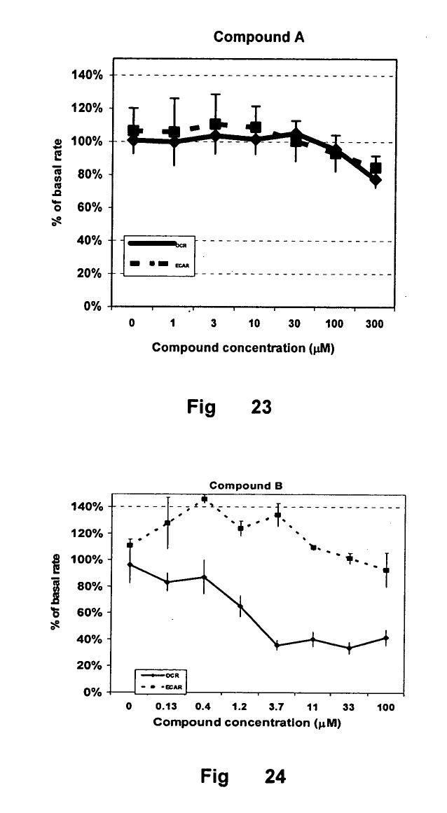

The highly metastatic tumor cell line, C₄₋₂ has a very low basal OCR/ECAR ratio demonstrating a highly glycolytic phenotype (FIG. 21). C₄₋₂ is derived from the non-invasive, immortalized LNCaP cell line. C₄₋₂ was exposed to increasing doses of topotecan, which inhibits HIF-1α, for 24 hours before XF measurement. Topotecan reduced the cellular rate of glycolysis and concomitantly stimulated the cellular rate of mitochondria respiration that quickly plateaued perhaps due to limited respiratory capacity of this highly aggressive tumor line (FIG. 22). Cells slowed proliferation but remained viable as confirmed by trypan blue staining. OCR and ECAR were normalized to rate per cell.

XF assay allows the direct and simultaneous detection of changes in glycolysis and mitochondrial respiration as a result of modulation of HIF-1α mediated oncogenic pathways.

### Example 8

### Discrimination of Functional Properties of Two Compounds Derived from a Common Parent

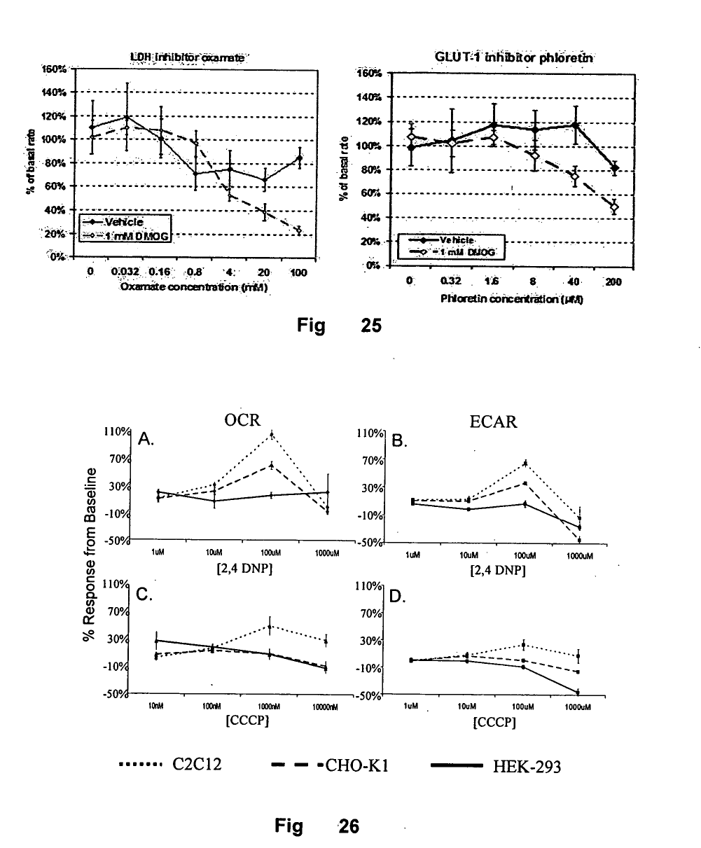

Two candidate drug compounds that were derived from a common parent and that demonstrated similar results in a conventional proliferation assay were assessed using the XF method. LNCaP cells were exposed to each compound, and then acute OCR and ECAR changes were measured as described above. As shown in FIG. 23, compound A modestly inhibited both OCR and ECAR, while compound B (FIG. 24) drastically decreased OCR in a dose dependent manner. The OCR and ECAR response to chronic treatment (>24 hours) was similar to the acute response.

This suggests that the compound series, of which A and B are members, have at least two activities, one that affects proton extrusion and the other oxygen consumption, and that the structure of compound B has enhanced effect on the oxygen consumption pathway. Since this difference was not visible using conventional proliferation assays, the results suggest that the XF method may be useful for supporting drug lead optimization, and may provide better prediction of in vivo function especially for these compounds as they are intended to directly affect cellular metabolism for a beneficial disease modulation.

### Example 9

### Altered Bioenergetic Profile in LNCaP Cells Treated with Dimethyloxalylglycine

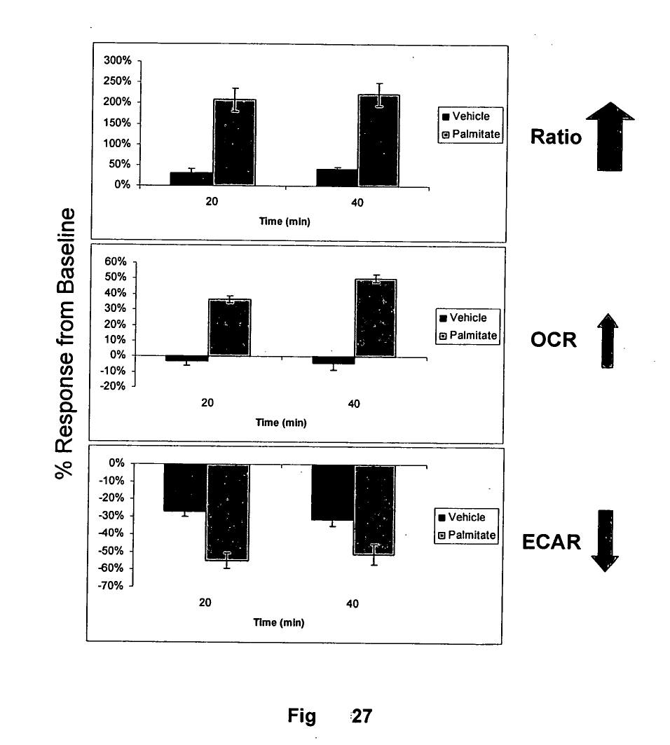

LNCaP cells were exposed to 1 mM DMOG, which acts to increase HIF-1α, for 24 hours prior to XF measurement. As demonstrated in FIG. 25, pretreatment with 1 mM DMOG versus vehicle control resulted in a greater decrease of ECAR in the presence of either oxamate (LDH inhibitor) or phloretin (GLUT1 inhibitor). Cells in both experiments continued normal proliferation and were viable as assessed by Trypan blue staining.

### Example 10

### Decreased Oxygen Consumption Rate in Colon Cancer Cells after 24 Hour Exposure to an Inhibitor of Proton Pump vATPase

Colon cancer cells were exposed to a vATPase inhibitor for 24 hours. OCR and ECAR were measured. The treated cells exhibited a dose-dependent OCR decrease after normalized to cell number. The inhibitor was shown to inhibit cell proliferation, but its effect on oxygen consumption was not known. vATPase inhibitors are being developed as anti-cancer drugs.

### Example 11

### Comparison of the Effects of Two Drugs that Induce Mitochondrial Uncoupling in Three Different Cell Lines in Order to Select an Appropriate System for Fatty Acid Oxidation Experiments

The purpose of this experiment was to compare the respiratory capacity of three distinct cell lines based on their response to two drugs that induce mitochondrial uncoupling (disruption of the electron transport chain). Depending on the type of biology or diseased to be modeled, there is a need for cells to have a relatively higher or lower potential for fatty acid oxidation. By profiling different cells lines and/or optimizing their culture media or environmental components one can select cell lines and/or conditions that are appropriate for the biological or disease model.

C2C12, CHO-K1, and HEK-293 cells were profiled using the Seahorse XF instrument to compare basal metabolic rates and respiratory pathway preference, and then to compare the effects of two uncouplers of mitochondrial respiration. Chinese hamster ovary cell line CHO-K1, mouse muscle myoblast cell line C2C12, and human embryonic kidney cell line Hek-293 were obtained from the ATCC (Manassas, Va.). CHO-K1 cells were maintained in Ham's F12K media supplemented with 10% fetal bovine serum (FBS) and 100 μg/ml penicillin-Streptomycin. C2C12 myoblast cells were maintained in DMEM media supplemented with 10% FBS and 100 μg/ml penicillin-Streptomycin. Hek-293 cells were maintained in MEM supplemented with 10% heat-inactivated horse serum and 100 μg/m penicillin-Streptomycin.

2,4-dinitrophenol and carbonyl cyanide m-chlorophenylhydrazone were prepared according to the manufacturers' instructions. Cells were treated with trypsin and resuspended in 1 ml of culture media. The number of viable cells was determined by using a ViCell automated Trypan blue counter (Beckman-Coulter).

For XF assays, adherent cells were seeded in 24 well cell culture microplates. CHO-K1 cells were seeded at 30,000 cells/well, C2C12 myoblasts were seeded at 45,000 cells/well, and HEK-293 cells were seeded at 60,000 cells/well. Approximately 45 minutes prior to the first measurement, the culture medium was exchanged with 900 μL of a low-buffered DMEM assay medium to ensure accurate ECAR readings for CHO-K1, C2C12 myoblast, and HEK-293 cells.

Metabolic flux rate measurements were performed using a prototype Seahorse XF instrument. Non-invasive measurements of OCR and ECAR were made every 8 seconds for a period of 5 minutes. During this time, the media dissolved oxygen concentration decreased approximately 10%, and the media pH decreased approximately 0.1 unit.

Baseline metabolic rates were measured twice, and were reported in nmol/min for OCR and mpH/min for ECAR. 100 μL of a 10× compound solution was then added to the media and mixed for 5 minutes, and then the post-treatment OCR and ECAR measurements were made and repeated once. At the completion of each assay, cells were suspended from the well bottoms using trypsin, and a cell number count was obtained using the ViCell instrument.

FIG. 26 shows the resulting changes in OCR and ECAR in three distinctly different cell types upon challenge with mitochondrial uncoupling agents. Each cell type's OCR and ECAR was measured before and after addition of either uncoupling agent. Data are expressed as a percentage change in OCR or ECAR from each cell types' basal behavior. Panel (A) shows the OCR response of each cell type to the uncoupler 2,4 DNP whereas panel (B) shows the simultaneous change in ECAR. Panel (C) shows the OCR response of each cell type to the uncoupler CCCP whereas panel (D) shows the simultaneous change in ECAR. The data shows that C2C12 myoblasts have a higher excess respiratory capacity than the less metabolically active CHO-K1 and HEK-293 cells, and therefore have a higher overall FAO potential.

### Example 12

### Measurement of Stimulated Fatty Acid Oxidation in C2C12 Myocytes

C2C12 myoblasts were seeded at a density of 30,000 cells/well in a 24 well cell culture microplate containing DMEM maintenance medium as described in example 11, but using 2% FBS. After seven days, myotube formation was visible microscopically.

Palmitate was prepared using the following procedure. 0.4 mM FAF-BSA/KHB solution was dialyzed against KHB to remove calcium from the BSA. Palmitic acid (20 mM) was then dissolved in 100% ETOH and an aliquot placed in a 16×100 mm glass tube. The ethanol was then removed under nitrogen. 0.5 ml of dialyzed FAF-BSA complex was then added to the 20 mM dried aliquot, and the resulting mixture was heated at 37° C. for 1 hour with frequent mixing.

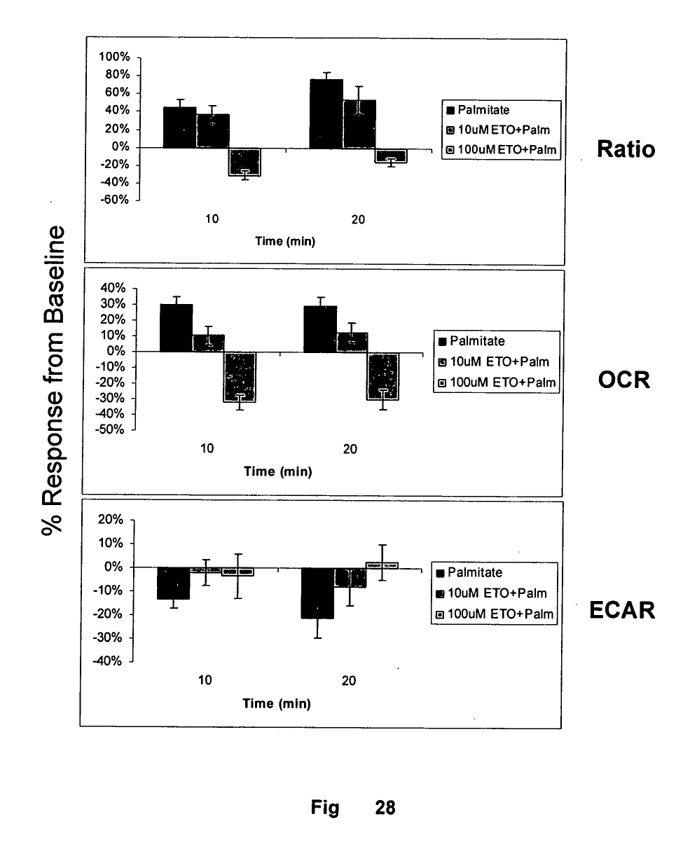

C2C12 myocytes were prepared for testing by replacing the differentiation medium with 900 μL of Krebs Henseleit Buffer (KHB without calcium). After a baseline XF reading was obtained, cells were challenged with either 0.4 mM FAF-BSA vehicle alone, or 200 μM palmitate complexed with 0.4 mM FAF-BSA. Metabolic flux measurements were taken as described in Example 1. OCR/ECAR ratios were calculated based on raw values without normalization for cell number. The resulting increase in the ratio of OCR/ECAR upon addition of palmitate is shown in FIG. 27 and is indicative of the increase in oxygen consumption relative to proton secretion that is characteristic of increased fatty acid oxidation over glycolysis.

### Example 13

### Confirmation of Fatty Acid Oxidation Measurements Using Inhibitors

The CPT-1 inhibitor Etomoxir was prepared according to the manufacturer's instructions. Palmitate was prepared as described in example 12. Using the experimental protocol of example 12, C2C12 myocytes were incubated with palmitate and/or the Etomoxir. OCR and ECAR were then measured in the prototype Seahorse XF instrument for 40 minutes (FIG. 28).

Etomoxir produced a significant reduction in OCR (middle panel) and an increase in ECAR (bottom panel) indicating a change from fatty acid to glucose utilization. This is reflected in the greatly reduced OCR/ECAR ratio (top panel) post exposure to 100 μM Etomoxir. It should be noted that all data were normalized against vehicle prior to calculating percent change from baseline. OCR/ECAR percent increase above baseline is shown in the top panel, percent increase in OCR and ECAR are shown in the middle and bottom panels, respectively.

### Example 14

### Enhancement of Fatty Acid Oxidation Using Agonists

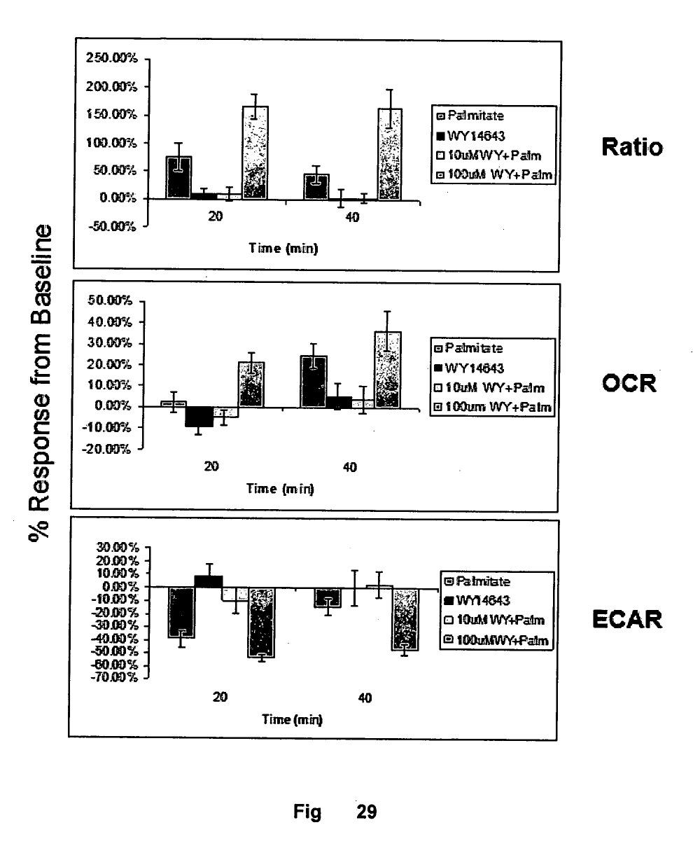

Using the experimental protocol of example 12, C2C12 myocytes were incubated with palmitate and/or the PPARα agonist WY14643. (PPARα is a gene that is the basis of Thiazolidinediones.) OCR and ECAR were then measured in the prototype Seahorse XF instrument for 40 minutes (FIG. 29).

WY14643 produced a significant increase in OCR (middle panel) and a decrease in ECAR (bottom panel) indicating a significant enhancement of fatty acid oxidation. This is reflected in the greatly increased OCR/ECAR ratio (top panel) post exposure to 100 μM WY14643. All data was normalized against vehicle, prior to calculating percent change from baseline. OCR/ECAR percent increase above baseline is shown in the top panel, percent increase in OCR and ECAR are shown in the middle and bottom panels, respectively.

### Example 15

### Assessment of Cell Quality Using Measurement of Extracellular Flux Rates

The purpose of this experiment was to demonstrate that extracellular flux rate measurements can provide indication of poor cell quality/vitality. Myoblast cells were degraded by brief exposure to elevated temperature, and cells were then profiled using a conventional viability assay (Trypan blue dye exclusion) and an extracellular flux rate measurement. The results were then compared.

CHO cells (CHO-M3) were obtained from the ATCC. Cells were seeded at 200,000 cells/ml the day before the experiment in a 24-well plate. Cells were cultured, in T175 flasks at 37 degrees C., in a humidified chamber, in 5% CO₂, using HAMS F-12 Media (ATCC) containing 10% FBS (ATCC Part# 30-2003), 100 units/ml and 100 μg/ml, respectively, of Pen. Strep, 2 mM Glutamax, 1 mM Sodium Pyruvate, and 0.1 mg/ml G418 selector. Cells were passaged every 48 hours or at 70% confluency. Cells were passaged by removing media, rinsing with 5 ml PBS (CellGro Part# 21-030-CV), and incubating each flask at 37 degrees C. for 2 minutes with 2 ml trypsin. Immediately following incubation with trypsin, 8 ml of media were added with swirling. Cells were triturated >10 times using a 5 ml pipette with the pipette tip being placed in the lowest corner of the flask. Cells and media were transferred to 50 ml centrifuge tubes and triturated 10-15 times to insure a homogeneous mixture of cells. Following detachment, cells were either passaged to new T175 flasks and split at a ratio of 1:6-1:10 or seeded in 24-well plates at a density of 150,000 cells/ml in each well. Cells seeded in 24 well formats were grown for 24 hours prior to using them in any assay.

Cells were exposed to elevated temperatures of 50, 57, or 65 degrees C. for ten minutes. Within ten minutes of heat exposure, cell growth media was replaced with low buffered media (Sigma D-5030), and extracellular flux rates (OCR and ECAR) were measured using the prototype Seahorse XF instrument. Following these measurements, cell viability was measured using a Beckman Coulter Vi-Cell Cell Viability Analyzer. This is an automated, flow through, imaging system that uses the Trypan blue cell exclusion stain method. Cell viability was monitored, at least representatively, on every day that tests were performed.

As shown in FIG. 30, cellular OCR and ECAR readings dropped significantly, and in a linearly increasing manner following exposure to increasing temperatures, indicating damage to the respiratory mechanisms of the cells. Vitality measurement declined only at the higher temperature points.

This demonstrates the sensitive detection of cellular damage that can be obtained using acute extracellular flux measurements, and the improved detection capability over the traditional membrane integrity assay.

### Example 16

### Optimization of Cell Behavior Using Measurement of Extracellular Flux Rates

The purpose of this experiment was to optimize the level of FAO induction by palmitate to enable a robust agonist response using the AMP analog AICAR. The desired outcome is to better predict in vivo induction of FAO by various agonist compounds. Several concentrations of glucose (0 mM, 1 mM, 2.5 mM, 5.0 mM and 10 mM) were added to the culture medium to condition the C2C12 myocytes. As well, the cells were exposed to different concentrations of Palmitate (50 μM, 100 μM, 150 μM and 200 μM). While certain concentrations of glucose and palmitate resulted in higher induction of FAO, 2.5 mM glucose and 150 μM Palmitate gave the optimal agonist response to AICAR These data (not shown) demonstrate the ability of extracellular flux measurements to enable the appropriate conditioning of cells to generate more relevant models of biology and disease for research and drug discovery. These may also be better predictors of in vivo efficacy.

### Example 17

### Assessment of the Potential Toxicity of a Candidate Drug Compound

The purpose of this experiment was to assess the potential toxicity of a candidate drug compound. A proprietary drug candidate, A-XXX, was chosen because it has previously been shown to exhibit toxicity in in vivo studies.

Hep-G2 cells (ATCC Part#HB-8065) were cultured in T75 flasks at 37 degrees C., in a humidified chamber, in 5% CO₂, using MEM (ATCC Part# 30-2003) containing 10% FBS, 100 units/ml and 100 μg/ml, respectively, of Pen. Strep., 2 mM Glutamax, and 1 mM Sodium Pyruvate (Sigma Part# S8636). Cells were passaged every 72 hours or at 80% confluency. Cells were passaged by removing media, rinsing with 5 ml PBS, and incubating each flask at 37 degrees C. for 2 minutes with 2 ml trypsin (Gibco Part#25200-072). Immediately following incubation with trypsin, flasks were gently tapped to insure complete cell detachment, and 8 ml of media were added with swirling. Cells were triturated >10 times using a 5 ml pipette with the pipette tip being placed in the lowest corner of the flask.

Cells and media were then transferred to 50 ml centrifuge tubes and triturated 10-15 times to insure a homogeneous mixture of cells. Following detachment, cells were either passaged to new T75 flasks and split at a ratio of 1:3 or seeded in 24-well plates at a density of 220,000 cells/ml in each well. Cells seeded in 24 well formats were grown for 48 hours prior to using them in any assay.

On the day of experiment, cell growth media was replaced with low buffered media (Sigma D-5030), and extracellular flux rates (OCR and ECAR) were measured using the prototype Seahorse XF instrument. Following these measurements, cell viability was measured using a Beckman Coulter Vi-Cell Cell Viability Analyzer.

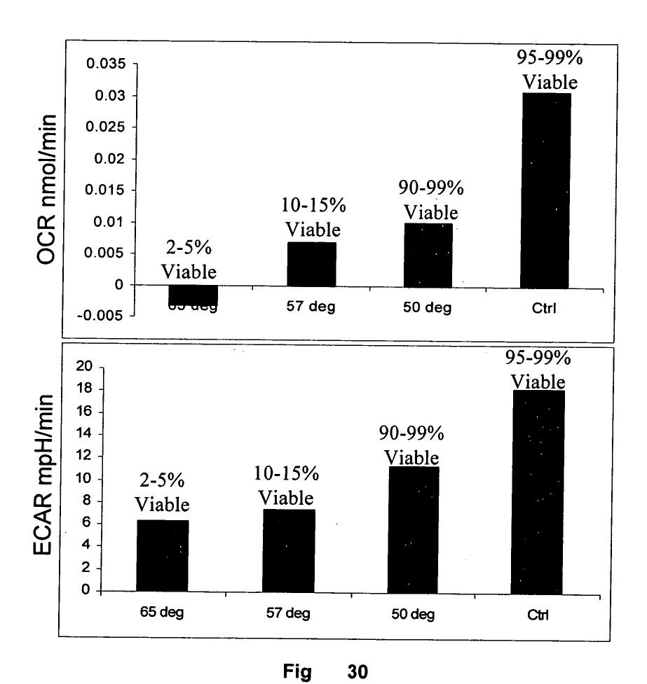

Cell counts were acquired using a Vi-Cell Cell Viability Analyzer on every day that tests were performed. The drug compound had no significant effect on cell viability at any of the concentrations used. All viability measurements were made following a total of 40 minutes of exposure to the compound. Furthermore, percent cell viabilities at various concentrations of each compound ranged from 90% viable to 96% viable. The mean viability across all concentrations of all compounds shown in FIG. 31 was 94%±1% that being nearly identical to the percent viability normally observed in stock flasks and control wells (i.e. 95% [data not shown]).

As shown in FIG. 31, compound A-XXX elicited a dose dependent decrease in OCR as measured by the rate of O₂ consumption in the media. The rate of pH change indicated a dose dependent increase in ECAR which was inversely related to the change in OCR. Maximal inhibition of cellular respiration (52%) occurred at the highest dose (100 μM). At this same compound concentration, there was a 63% average increase in the rate of ECAR. Vehicle (DMSO control) treated Hep-G2 cells show essentially no change in either OCR or ECAR (5% or less).

The inversely related increase in ECAR and decrease in cellular respiration are potentially explained by compound A-XXX's mechanism of action. Rotenone, a mitochondrial complex I inhibitor toxin has been shown to decrease OCR (as measured by O₂ rate), but to have no effect on ECAR (as measured by pH rate) [Seahorse Data, Data not shown]. Rotenone's inhibition at the complex I portion of the electron transport chain leaves alternate oxidative processes open through complex II.

We propose that A-XXX inhibits both complexes I and II and therefore shuts down oxidative metabolism more completely, thereby driving glycolytic processes. As glycolytic processes are known to generate much higher ECAR rates per ATP produced due to lactate accumulation, this may explain the dose dependent changes in ECAR seen with A-XXX.

### Example 18

### Assessment of the Percent Uncoupled Metabolism

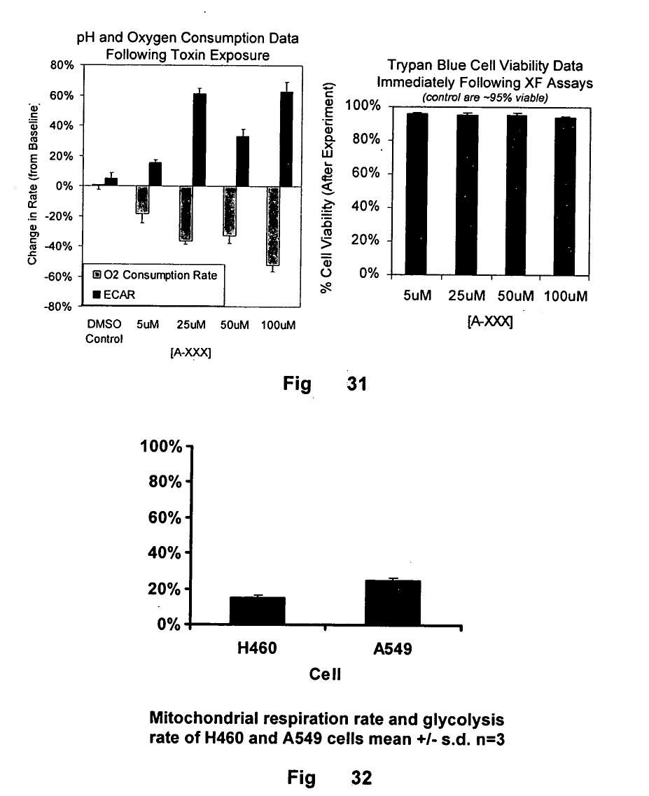

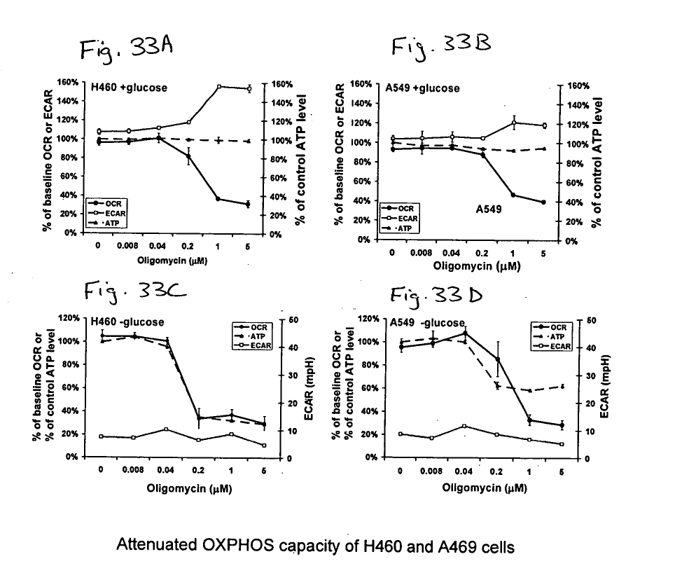

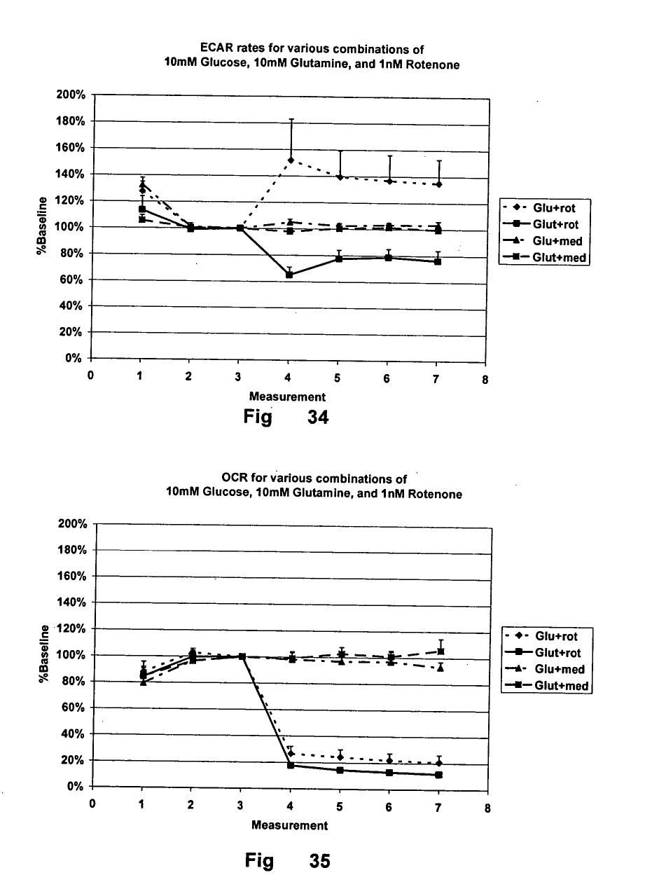

Mitochondrial proton leak. It is well established that mitochondrial respiration is comprised of coupled and uncoupled respiration. Coupled respiration represents the fraction that is used for ATP synthesis, while uncoupled respiration represents the fraction of mitochondrial respiration that is used to drive the futile cycle of proton pumping and proton leak back across the inner mitochondrial membrane. Cells or tissues derived across animal species in vitro spend up to 20% of their basal mitochondrial respiration rate to drive proton leak and the remaining 80% is coupled to ATP turnover. The proposed physiological function of proton leak includes heat production and prevention of oxidative stress caused by reactive oxygen species. Uncoupled and coupled respiration was calculated for two cell lines, H460 and A549 with data shown in FIGS. 32 and 33A-33D. FIG. 32 is a bar graph showing mitochondrial respiration rate and glycolysis rate of H460 and A549 cells. (Mitochondrial respiration rate, rotenone-sensitive total respiration. Results are expressed as mean±SD, n=3.) In FIG. 32, mitochondrial respiration of H460 and A549 cells account for approximately 85% and 75% of total cellular respiration, respectively. The coupled respiration and proton leak were determined using the ATP synthase inhibitor oligomycin as shown in FIG. 33. The oligomycin-sensitive respiration was ˜70% and ˜60% of the total cellular respiration rate in H460 and A549 cells respectively (FIGS. 33A and 33B). Thus, H460 and A549 cells devoted ˜82.5% and ˜80% of its mitochondrial respiration for ATP turnover and ˜17.5% and 20% for proton leak, respectively. A similar distribution of ATP turnover and proton leak was maintained in the glucose deprived medium (FIGS. 33C and 33D) assuming the non-mitochondrial respiration was similar in both glucose containing and deprived medium. In the presence of glucose (A and B), FIFO ATP synthase inhibitor oligomycin, suppressed respiration and stimulated glycolysis whereas ATP turn over was sustained. In the absence of glucose (C and D), oligomycin caused reduction in respiration as well as ATP level while glycolysis rate was not increased even slightly decreased. Low buffered RMPI 1640 assay medium contains 11 mM glucose and 2 mM L-glutamine. Glucose-deprived assay medium consists of low buffered KHB supplement with 10 mM L-glutamine. Results are expressed as mean±SD. n=3 except ECAR in C and D. Note, in C &D, ECAR was presented as absolute mean value in contrast with percentage of control ECAR in A and B illustrate the very low rate (noisy), a true effect of glucose-deprivation which would have been misleading if presented as percentage of baseline.

### Example 19

Experiments were performed to determine the amount of activity in the HMP pathway by comparing the production of CO₂ of cells in media containing glucose (produces CO₂ when catabolized) to the production of CO₂ of cells in media containing glutamine (does not produce CO₂ when catabolized), while exposed to rotenone. Rotenone is a complex one respiratory inhibitor that acts to block NADH dehydrogenase thereby interrupting the process of aerobic metabolism within the mitochondria. During this experiment three separate analytes were measured simultaneously providing a kinetic image of: inhibition of the oxidative respiration; activation of anaerobic glycolysis, and; a substantial increase in HMP activity

C2C12 fibroblast cells were seeded at 40K cells per well and incubated for 30 minutes prior to the assay in media (Krebs KHB buffer) containing either 10 mM glucose or 10 mM glutamine. Each subgroup of cells then were either exposed to 1 nM rotenone or media.

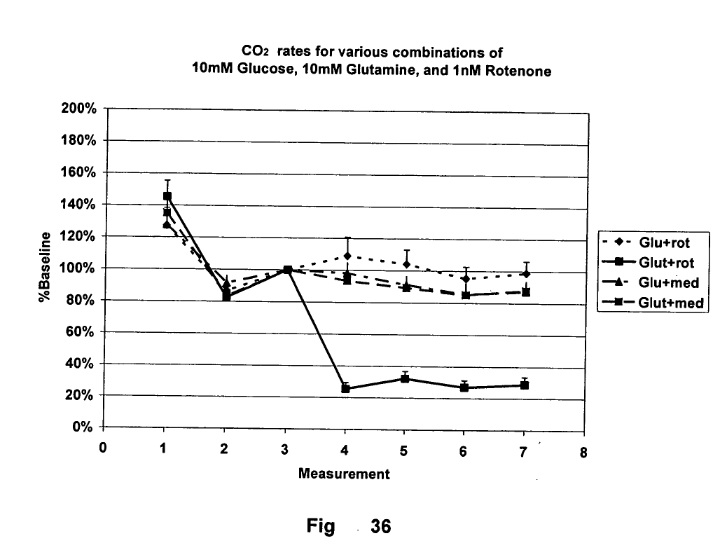

Upon exposure to the 1 nM rotenone there is a significant inhibition of oxygen consumption (OCR) in both cell populations, as shown in FIG. 34. Upon exposure to 1 nM rotenone cells in media containing glutamine show a 45% decrease in ECAR as compared to cells in media containing glucose which show a 75% increase in ECAR (see FIG. 35) The increase in ECAR is likely due to the contribution of lactic acid as the cells switch to anaerobic glycolysis and increased activity in the HMP pathway increasing carbonic acid production. The noted decrease in CO₂ of cells in glutamine is consistent with the expected decrease in CO₂ production as a result of inhibition in the respiratory pathway with rotenone exposure.

Upon exposure to 1 nM rotenone cells in media containing glutamine show a >50% decrease in CO₂ production while cells in media containing glucose show a slight increase in CO₂ production (see FIG. 36). This clearly shows the utility of the CO₂ sensor and its ability separate HMP activity as this is the only remaining pathway (in the absence of the Kreb cycle) that can pass CO₂ to the surrounding media. A requirement of this pathway is the presence of glucose.

### INCORPORATION BY REFERENCE

The entire content of each patent and non-patent document disclosed herein is expressly incorporated herein by reference for all purposes.

### EQUIVALENTS

The invention may be embodied in other specific forms without departing from the spirit pr essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A method for analysis comprising the steps of:
incubating animal cells under analysis in a medium disposed in at least one of a plurality of wells in a multiwell plate;
adding to the medium to bring into contact with the cells a substance potentially capable of altering cellular metabolism; and
measuring in medium in a well the rate of change in concentration of both an extracellular solute which is a component of cellular aerobic metabolism and an extracellular solute which is a component of cellular anaerobic metabolism.

2. The method of claim 1 wherein the animal cells are primary.

3. The method of claim 1 wherein the animal cells are neoplastic.

4. The method of claim 1 wherein the animal cells are adherent to a substrate.

5. The method of claim 1 wherein the animal cells are in suspension.

6. The method of claim 1 wherein the substance is a drug candidate.

7. The method of claim 1 wherein the substance is a toxin.

8. The method of claim 1 wherein the substance is a ligand known or suspected to bind to a cell surface receptor.

9. The method of claim 1 wherein the measurements are conducted substantially simultaneously in said well.

10. The method of claim 1 comprising the additional steps of:
measuring, in said cell medium prior to addition of said substance, or in the medium of a cell culture in a well separate from said cells under analysis, the rate of change in concentration of the extracellular solute which is a component of cellular aerobic metabolism and the extracellular solute which is a component of cellular anaerobic metabolism, and
comparing the said rates of change prior to and after addition of said substance to assess the effect of said substance on the metabolic activity of said cells.

11. The method of claim 1 comprising the additional step of:
incubating said cells in the presence of said substance for a predetermined time interval prior to measuring said rates of change.

12. The method of claim 111 comprising the additional steps of:
measuring, in the medium of a cell culture in a well separate from said cells under analysis and treated differently than said cells under analysis, either or both the rate of change in concentration of an extracellular solute which is a component of cellular aerobic metabolism and an extracellular solute which is a component of cellular anaerobic metabolism; and
comparing the measurements of the rate of change in said separate cell culture to said cells under analysis.

13. The method of claim 1 comprising the additional steps of:
adding to the medium in separate cultures of the same cells in different wells different concentrations of said substance potentially capable of altering cellular metabolism; and
measuring in the cell medium in said separate cultures said rates of change.

14. The method of claim 1 comprising the additional step of:
measuring in the cell medium the rates of change in concentration at different times to obtain a temporal profile of the effect of said substance on said cells.

15. The method of claim 1 comprising adding a fatty acid to a said well to assess a characteristic of fatty acid metabolic activity of a said cell culture.

16. The method of claim 1 wherein said component of cellular aerobic metabolism is extracellular oxygen and said measurement is oxygen consumption rate.

17. The method of claim 1 wherein said component of cellular anaerobic metabolism is extracellular proton concentration and said measurement is the extracellular acidification rate.

18. The method of claim 1 wherein said component of cellular aerobic metabolism is the extracellular concentration of carbon dioxide and said measurement is the extracellular carbon dioxide production rate.

19. The method of claim 1 wherein said component of cellular anaerobic metabolism is the extracellular concentration of either lactic acid or lactate.

20. The method of claim 1 comprising incubating in parallel plural cultures of said animal cells in plural wells, adding to the media in different wells different substances or different concentrations of the same substance, and measuring said rate of change in plural wells.

21. The method of claim 1 wherein the step of measuring the rate of change in concentration in a cell medium comprises the step of temporarily reducing the volume of medium in a cell culture to increase the sensitivity of solute concentration changes.

22. The method of claim 1 comprising, prior to adding said substance, measuring in a cell medium in a well the rate of change in concentration of both an extracellular solute which is a component of cellular aerobic metabolism and an extracellular solute which is a component of cellular anaerobic metabolism, adding to the medium a drug that increases cellular metabolism, and measuring in the medium the rates of change in concentration of plural extracellular solutes, one of which is a component of cellular aerobic metabolism and another of which is a component of cellular anaerobic metabolism, and comparing the measurement, thereby to measure the relative excess metabolic capacity of said cells.

23. A method for analysis of cell culture quality comprising the steps of:
measuring in a cell medium the rate of change in concentration of both an extracellular solute which is a component of cellular aerobic metabolism and an extracellular solute which is a component of cellular anaerobic metabolism; and
comparing said measured rates of change to a standard informative of known cell culture respiration rates thereby to assess the respiratory capacity, metabolic rate, or relative pathway utilization of the culture as a measure of cell vitality and cell quality.

24. The method for analysis of claim 23 comprising comparing said measured rates of change to rates measured in a culture comprising a known number of healthy cells of a cell type having inherently comparable metabolic rates or pathway utilization properties to the cells under quality assessment.

25. The method for analysis of claim 23 comprising seeding cells at a predetermined density in a test well prior to said measuring step thereby to enable direct comparison of said measured rates of change to a standard.

26. The method of claim 23 wherein said component of cellular aerobic metabolism is extracellular oxygen and said measurement is oxygen consumption rate.

27. The method of claim 23 wherein said component of cellular anaerobic metabolism is extracellular proton concentration and said measurement is the extracellular acidification rate.

28. The method of claim 23 wherein said component of cellular aerobic metabolism is the extracellular concentration of carbon dioxide.

29. The method of claim 23 wherein said component of cellular anaerobic metabolism is the extracellular concentration of lactic acid or lactate.

30. The method of claim 15 comprising the additional step of:
incubating said cells in a drug prior to adding a fatty acid to a said well to assess any effect on fatty acid metabolism.

31. The method of claim 30 comprising the additional step of
adding a substance known to inhibit fatty acid transport or oxidation to medium in a said well in order to determine more specifically the effect of said drug.

32. A method for analysis comprising the steps of:
a) exposing an animal to a test substance;
b) removing cells from said animal;
c) incubating said cells in a medium disposed in at least one of a plurality of wells in a multiwell plate;
d) measuring in the medium in the well the rate of change in concentration of both an extracellular solute which is a component of cellular aerobic metabolism and an extracellular solute which is a component of cellular anaerobic metabolism.

33. The method of claim 32 further comprising the additional steps of:
e) repeating steps b), c), and d), and;
f) comparing said measured rates produced in different cycles.

34. A method for analysis comprising the steps of:
incubating animal cells under analysis in a medium disposed in at least one of a plurality of wells in a multiwell plate;
subjecting said cells to a genetic alteration or environmental stress potentially capable of altering cellular metabolism; and
measuring in medium in a well the rate of change in concentration of both an extracellular solute which is a component of cellular aerobic metabolism and an extracellular solute which is a component of cellular anaerobic metabolism.
